(19) United States
(12) Patent Application Publication
(10) Pub. No.: US 2022/0119794 A1
(43) Pub. Date: Apr. 21, 2022

(54) CHEMICALLY PROGRAMMED NEUTROPHILS AND USES THEREOF

(71) Applicant: VIRGINIA POLYTECHNIC INSTITUTE AND STATE UNIVERSITY, Blacksburg, VA (US)

(72) Inventors: Liwu LI, Blacksburg, VA (US); Shuo GENG, Blacksburg, VA (US); Yao ZHANG, Blacksburg, VA (US)

(21) Appl. No.: 17/419,274

(22) PCT Filed: Dec. 23, 2019
(86) PCT No.: PCT/US2019/068448

§ 371 Date: Jun. 28, 2021

Related U.S. Application Data
(60) Provisional application No. 62/785,459, filed on Dec. 27, 2018.

(51) Int. Cl. C12N 15/01; C12N 15/85; A61K 35/15; A61K 31/192; A61P 29/00; A61P 9/10

(52) U.S. Cl. CPC C12N 15/01; C12N 15/85; A61P 9/10; A61K 31/192; A61P 29/00; A61K 35/15

(57) ABSTRACT

Described herein are embodiments of chemically modified neutrophils and pharmaceutical formulations thereof. Also described herein are 4-phenylbutyrate pharmaceutical formulations. Also described herein are methods of chemically modifying neutrophils. Also described herein are treatments for non-resolving inflammation and/or related diseases or conditions, including but not limited to atherosclerosis, cardiovascular disease, stroke, myocardial infarction, neurological disease, and/or a symptom thereof in a subject in need thereof.

### CROSS-REFERENCE TO RELATED APPLICATIONS

This application claims the benefit of and priority to co-pending U.S. Provisional Patent Application No. 62/785,459, filed on Dec. 27, 2018 entitled “CHEMICALLY PROGRAMMED NEUTROPHILS AND USES THEREOF,” the contents of which is incorporated by reference herein in its entirety.

### STATEMENT REGARDING FEDERALLY SPONSORED RESEARCH

This invention was made with Government support HL115835 awarded by the National Institute of Health. The Government has certain rights in the invention.

### TECHNICAL FIELD

The subject matter disclosed herein is generally directed to programming immune cells, such as neutrophils.

### BACKGROUND

Atherosclerosis is generally the hardening and narrowing of arteries and can lead to artery blockage, which can result in heart attacks, strokes, and peripheral vascular diseases and conditions. Atherosclerosis can be caused in part by the build-up of plaque on the walls of the vessels. While some treatments and preventions exist, atherosclerosis is still a significant health issue and causes a significant amount of mortality and morbidity. As such there exists a need for compositions and techniques to treat and/or prevent atherosclerosis. Citation or identification of any document in this application is not an admission that such a document is available as prior art to the present invention.

### SUMMARY

In some exemplary embodiments, described are chemically programmed neutrophils that can be characterized in that they have increased surface expression of CD62L as compared to a control, decreased gene and/or protein expression of CD11b, MMP-9, LTB4, MPO, and/or Dectin-1 as compared to the control, increased gene and/or protein expression of FPN, TGFβ, and/or LRRC32 as compared to the control, decreased activity of oxCAMKII as compared to the control, or any combination thereof, wherein the control is a neutrophil or population thereof having non-resolving inflammation phenotype. In some exemplary embodiments, further comprises an exogenous gene. In some exemplary embodiments, the exogenous gene is a selectable marker or a suicide gene. In some exemplary embodiments, the chemically programmed neutrophil was made by the method comprising: contacting a neutrophil with 4-phenylbutyrate. In some exemplary embodiments, the concentration of 4-phenylbutyrate ranges from about 0.1 mM to about 10 mM. In some exemplary embodiments, the concentration of 4-phenylbutyrate is about 1 mM. In some exemplary embodiments, the step of contacting occurs ex vivo. In some exemplary embodiments, the step of contacting occurs in vivo.

In some exemplary embodiments, described herein are pharmaceutical formulations composed of a chemically programmed neutrophil or population thereof as described herein; and a pharmaceutically acceptable carrier.

In some exemplary embodiments, described herein are methods composed of administering a chemically programmed neutrophil as described herein or a population thereof or a pharmaceutical formulation thereof to a subject.

In some exemplary embodiments, the subject has or is suspected of having non-resolving inflammation and/or related diseases or conditions, including but not limited to atherosclerosis, cardiovascular disease, stroke, and myocardial infarction and/or a symptom thereof.

In some exemplary embodiments, described herein are methods of treating and/or preventing non-resolving inflammation and/or related diseases or conditions, including but not limited to atherosclerosis, cardiovascular disease, stroke, myocardial infarction, a neurological disease, and/or a symptom thereof in a subject in need thereof, the method comprising administering a chemically programmed neutrophil as described herein or a population thereof or a pharmaceutical formulation thereof to the subject in need thereof.

In some exemplary embodiments, described herein are methods of reducing arterial plaque in a subject in need thereof, the method comprising: administering a chemically programmed neutrophil as in described herein or a population thereof or a pharmaceutical formulation thereof to the subject in need thereof.

In some exemplary embodiments, are methods of chemically programming a neutrophil, the method comprising: contacting a neutrophil with 4-phenylbutyrate. In some exemplary embodiments, the concentration of 4-phenylbutyrate ranges from about 0.1 mM to about 10 mM. In some exemplary embodiments, the concentration of 4-phenylbutyrate is about 1 mM. In some exemplary embodiments, the step of contacting occurs ex vivo.

In some exemplary embodiments, the method further includes expressing an exogenous gene in the neutrophil. In some exemplary embodiments, the method further includes the step of harvesting the neutrophils from a subject, wherein the step of harvesting occurs before the step of contacting. In some exemplary embodiments, the method further includes the step of administering the chemically programmed neutrophil to the subject. In some exemplary embodiments, the step of contacting occurs in a subject. In some exemplary embodiments, the step of administering an amount of 4-PBA or a pharmaceutical formulation thereof to the subject, wherein the step of administering occurs before the step of contacting.

In some exemplary embodiments, described herein are pharmaceutical formulations for treating and/or preventing non-resolving inflammation and/or related diseases or conditions, including but not limited to atherosclerosis, cardiovascular disease, stroke, myocardial infarction, a neurological disease, and/or a symptom thereof in a subject in need thereof comprising: a therapeutically effective amount of 4-phenylbutyrate or pharmaceutically acceptable salt thereof; and a pharmaceutically acceptable carrier.

In some exemplary embodiments, described herein are methods of treating and/or preventing non-resolving inflammation and/or related diseases or conditions, including but not limited to atherosclerosis, cardiovascular disease, stroke, myocardial infarction, a neurological disease, and/or a symptom thereof in a subject in need thereof, the method comprising: administering a pharmaceutical formulation as described herein to the subject in need thereof.

### BRIEF DESCRIPTION OF THE DRAWINGS

Further aspects of the present disclosure will be readily appreciated upon review of the detailed description of its various embodiments, described below, when taken in conjunction with the accompanying drawings.

FIGS. 1A-1D can demonstrate that subclinical endotoxemia exacerbates atherosclerotic pathogenesis. ApoE−/− mice were administrated with PBS or super-low-dose LPS together with HFD for 4 weeks. (FIG. 1A) Representative images of HE-stained atherosclerotic lesions and quantification of plaque size demonstrated as the percentage of lesion area within aortic root area. Scale bar, 300 μm. (FIG. 1B) Representative images of Oil Red 0-stained atherosclerotic plaques and quantification of lipid deposition within lesion area. Scale bar, 300 μm. (FIG. 1C) Representative images of Picrosirius Redstained atherosclerotic plaques and quantification of collagen content within lesion area. Scale bar, 100 μm. (FIG. 1D) Determination of circulating MMP9, LTB4 and TGFβ levels by ELISA. Data are representative of two independent experiments, and error bars represent means±s.e.m. *P<0.05, **P<0.01 and ***P<0.001; Student's t-test (n=5 for each group).

FIGS. 2A-2C can demonstrate the subclinical endotoxin primes neutrophils into a pro-inflammatory state in atherosclerotic mice. ApoE−/− mice were administrated with PBS or super-low-dose LPS together with HFD for 4 weeks. The surface phenotypes of Ly6G+ neutrophils in the peripheral blood (FIG. 2A), bone marrow (FIG. 2B) and spleen (FIG. 2C) were analyzed with flow cytometry. Data are representative of two independent experiments, and error bars represent means±s.e.m. *P<0.05, **P<0.01 and ***P<0.001; Student's t-test (n=5 for each group).

FIGS. 3A-3F can demonstrate that super-low-dose LPS induces inflammatory polarization of neutrophils in vitro. Neutrophils were purified from the bone marrow of wild-type C57 BL/6 mice and treated with PBS, super-low-dose LPS (100 pg/ml) and/or oxLDL (10 μg/ml) for 2 d. (FIG. 3A) Levels of MMP9, LTB4, and MPO were determined by ELISA (n=3 for each group). (FIG. 3B) The surface phenotype of neutrophils was analyzed by flow cytometry (n=3 for each group). (FIG. 3C) Levels of miR-24 and miR-126 were determined by real-time RT-PCR (n=4 for each group). (FIG. 3D) The expressions of oxCaMKII and 5-LOX were determined by Western blot. (FIG. 3E) The representative histogram and quantification of p-STAT1 level as determined by flow cytometry (n=3 for each group). (FIG. 3F) Representative histograms and quantification of ATF4 and KLF2 levels as determined by flow cytometry (n=5 for each group). Data are representative of three independent experiments, and error bars represent means±s.e.m. *P<0.05, **P<0.01 and ***P<0.001; (a-c, e) one-way ANOVA; (0 Student's t-test.

FIGS. 4A-4E can demonstrate that neutrophils polarized by super low-dose LPS aggravate atherosclerosis. Neutrophils purified from ApoE−/− mice were treated with PBS or super-low-dose LPS (100 pg/ml) for 24 h. PBS- or LPS-polarized neutrophils (2×106 cells per mouse) were then adoptively transferred by intravenous injection to HFD-fed ApoE−/− mice once a week for 4 weeks. Samples were collected 1 week after the last neutrophil transfer. (FIG. 4A) Representative images of HE-stained atherosclerotic lesions and quantification of plaque size exhibited as the percentage of lesion area within aortic root area. Scale bar, 300 μm. (FIG. 4B) Representative images of Oil Red 0-stained atherosclerotic plaques and quantification of lipid deposition within lesion area. Scale bar, 300 μm. (FIG. 4C) Representative images of Picrosirius Red-stained atherosclerotic plaques and quantification of collagen content within lesion area. Scale bar, 100 μm. (FIG. 4D) Representative images and quantification of lesional oxCaMKII levels by confocal microscopy. Scale bar, 100 μm. (FIG. 4E) Determination of circulating MPO, MMP9 and LTB4 levels by ELISA. Data are representative of two independent experiments, and error bars represent means±s.e.m. *P<0.05, **P<0.01 and ***P<0.001; Student's t-test (n=5 to 6 for each group).

FIGS. 5A-5G can demonstrate that 4-PBA (Sodium phenylbutyrate) restores disrupted peroxisome homeostasis in neutrophils. Neutrophils were purified from the bone marrow of wild-type C57 BL/6 mice and treated with PBS, super-low dose LPS (100 pg/ml) and/or 4-PBA (1 mM) for 2 d. (FIG. 5A) Representative histogram of ROS level determined by CellROX labeling. (FIG. 5B) Quantification of ROS level in neutrophils (n=3 for each group). (FIG. 5C) Representative confocal microscopy images of the neutrophils stained with anti-PMP70 and anti-LAMP1 antibodies to demonstrate the localization and fusion of peroxisomes and lysosomes. Scale bar, 5 μm. (FIG. 5D) Western blot data of oxCaMKII and 5-LOX expression. (FIG. 5E) Representative histograms and quantification of p-STAT1 (n=3 for each group), ATF4 and KLF2 (n=5 for each group) levels as determined by flow cytometry. FIGS. 5F-5G can demonstrate that 4-PBA reverses LPS-induced neutrophil polarization. Neutrophils were purified from the bone marrow of wild-type C57 BL/6 mice and treated with PBS, super-low-dose LPS (100 pg/ml) and/or 4-PBA (1 mM) for 2 d. (FIG. 5F) The surface phenotype of neutrophils was analyzed by flow cytometry (n=3 for each group). (FIG. 5G) The levels of MPO, MMP9 and LTB4 were determined by ELISA (n=3 for each group). Data are representative of three independent experiments, and error bars represent means±S.E.M. **P<0.01 and ***P<0.001; one-way ANOVA.

FIG. 6 shows the levels of miR24 and miR126. The levels of miR-24 and miR-126 were determined by real-time RT-PCR (n=4 for each group). Data are representative of three independent experiments, and error bars represent means±S.E.M. *P<0.05, **P<0.01 and ***P<0.001; one-way ANOVA.

FIGS. 7A-7E can demonstrate that neutrophils reprogrammed by 4-PBA alleviate atherosclerosis. Neutrophils purified from ApoE−/− mice were treated with PBS or 4-PBA (1 mM) for 24 h. PBS- or LPS-polarized neutrophils (2×106 cells per mouse) were then adoptively transferred by intravenous injection to HFD-fed ApoE−/− mice once a week for 4 weeks. Samples were collected 1 week after the last neutrophil transfer. (FIG. 7A) Representative images of HE-stained atherosclerotic lesions and quantification of plaque size exhibited as the percentage of lesion area within aortic root area. Scale bar, 300 μm. (FIG. 7B) Representative images of Oil Red 0-stained atherosclerotic plaques and quantification of lipid deposition within lesion area. Scale bar, 300 μm. (FIG. 7C) Representative images of Picrosirius Red-stained atherosclerotic plaques and quantification of collagen content within lesion area. Scale bar, 100 μm. (FIG. 7D) Determination of circulating MPO, MMP9, LTB4 and TGF-β levels by ELISA. (FIG. 7E) Determination of circulating miR-24 and miR-126 levels by realtime RT-PCR. Data are representative of two independent experiments, and error bars represent means±s.e.m. *P<0.05, **P<0.01 and ***P<0.001; Student's t-test (n=5 to 7 for each group).

FIG. 8 can demonstrate that subclinical endotoxin up-regulates MPO level in HFD-fed mice. ApoE−/− mice were administrated with PBS or super-low-dose LPS together with HFD for 4 weeks. Circulating MPO level was determined by ELISA. Data are representative of two independent experiments, and error bars represent means±s.e.m. *P<0.05; Student's t-test (n=5 for each group).

FIG. 9 can demonstrate that subclinical endotoxemia exacerbates atherosclerotic pathogenesis in RD-fed mice. ApoE−/− mice were administrated with PBS or super-low-dose LPS together with RD for 4 weeks. Representative images of HE-stained atherosclerotic lesions and quantification of plaque size demonstrated as the percentage of lesion area within aortic root area (upper panels). Representative images of Oil Red 0-stained atherosclerotic plaques and quantification of lipid deposition within lesion area (middle panels). Representative images of Picrosirius Red-stained atherosclerotic plaques and quantification of collagen content within lesion area (lower panels). Scale bar, 300 μm (upper and middle panels) and 100 μm (lower panels). Data are representative of two independent experiments, and error bars represent means±s.e.m. **P<0.01; Student's t test (n=5 for each group).

FIG. 10 can demonstrate that subclinical endotoxemia exacerbates atherosclerotic pathogenesis in RD-fed mice. ApoE−/− mice were administrated with PBS or super-low-dose LPS together with RD for 4 weeks. Representative images of HE-stained atherosclerotic lesions and quantification of plaque size demonstrated as the percentage of lesion area within aortic root area (upper panels). Representative images of Oil Red 0-stained atherosclerotic plaques and quantification of lipid deposition within lesion area (middle panels). Representative images of Picrosirius Red-stained atherosclerotic plaques and quantification of collagen content within lesion area (lower panels). Scale bar, 300 μm (upper and middle panels) and 100 μm (lower panels). Data are representative of two independent experiments, and error bars represent means±s.e.m. **P<0.01; Student's t test (n=5 for each group).

FIGS. 11A-11C can demonstrate that subclinical endotoxin causes neutrophil expansion in atherosclerotic mice. ApoE−/− mice were administrated with PBS or super-low-dose LPS together with HFD for 4 weeks. The frequency of Ly6G+ neutrophils in the peripheral blood (FIG. 11A) and spleen (FIG. 11B) was analyzed with flow cytometry. (FIG. 11C) Representative confocal images and quantification of Ly6G+ neutrophils in atherosclerotic plaques. Scale bar, 100 μm. Data are representative of two independent experiments, and error bars represent means±S.E.M. *P<0.05; Student's t-test (n=5 to 6 for each group).

FIGS. 12A-12B can demonstrate polarization of neutrophils in vitro. Neutrophils were purified from the bone marrow of wild-type C57 BL/6 mice and treated with PBS, super-low-dose LPS (100 pg/ml) and/or oxLDL (10 μg/ml) for 2 d. (FIG. 12A) Production of MPO was determined by ELISA. (FIG. 12B) The surface expression of CD62L was analyzed by flow cytometry. Data are representative of three independent experiments, and error bars represent means±S.E.M. **P<0.01 and ***P<0.001; one-way ANOVA (n=3 for each group).

FIGS. 13A-13B can demonstrate that subclinical endotoxin induces oxCAMKII elevation in RD-fed mice. ApoE−/− mice were administrated with PBS or super-low-dose LPS together with RD for 4 weeks. Representative images and quantification of lesional oxCaMKII levels by confocal microscopy. Scale bar, 100 μm. (b) Determination of circulating MPO levels by ELISA. Data are representative of two independent experiments, and error bars represent means±S.E.M. *P<0.05; Student's t-test (n=5 for each group).

FIGS. 14A-14B can demonstrate that subclinical endotoxin induces oxCAMKII elevation in HFD-fed mice. ApoE−/− mice were administrated with PBS or super-low-dose LPS together with HFD for 4 weeks. Representative images and quantification of lesional oxCaMKII levels by confocal microscopy. Scale bar, 100 μm. Data are representative of two independent experiments, and error bars represent means±S.E.M. ***P<0.001; Student's t-test (n=5 for each group).

FIGS. 15A-15C can demonstrate subclinical endotoxin primes neutrophils into a proinflammatory state in atherosclerotic mice. ApoE−/− mice were administrated with PBS or super-low-dose LPS together with HFD for 4 weeks. The surface phenotypes of Ly6G+ neutrophils in the peripheral blood (FIG. 15A) spleen (FIG. 15B) and bone marrow (FIG. 15C) were analyzed by flow cytometry. The same data shown in FIGS. 2A-2C were re-analyzed with geometric mean fluorescence intensity (Geo MFI) as the parameter. Error bars represent means S.E.M. *P<0.05, **P<0.01 and ***P<0.001; Student's t-test (n=5 for each group).

FIGS. 16A-16E can demonstrate that neutrophils maintain viability after in vitro polarization. Bone marrow neutrophils were purified and treated with PBS, super-low-dose LPS (100 pg/ml) or 4-PBA (1 mM) for 24 h (FIGS. 16A-16B) and 48 h (FIGS. 16C-16D). The cells were stained with Annexin-V and PI, and the viability (percentage of Annexin-V-/PI-population) was determined by flow cytometry. (FIG. 16E) Surface phenotype of neutrophils was analyzed by flow cytometry after polarization for 24 h. Data are representative of three independent experiments, and error bars represent means±S.E.M. *P<0.05, **P<0.01 and ***P<0.001 as compared with PBS group; one-way ANOVA. (n=5 for each group).

FIGS. 17A-17D can demonstrate that transfusion of superlow-dose LPS-polarized neutrophils elevates plasma lipid levels and modulates lesional macrophages. Neutrophils purified from ApoE−/− mice were treated with PBS or super-low-dose LPS (100 pg/ml) for 24 h. PBS- or LPS-treated neutrophils (2×106 cells per mouse) were then adoptively transferred by intravenous injection to HFD-fed ApoE−/− mice once a week for 4 weeks. (FIG. 17A) Plasma samples were collected 1 week after the last neutrophil transfer, and the levels of total cholesterol, free cholesterol and triglycerides were determined. (FIG. 17B) Representative confocal images of CD68 and SR-A staining in atherosclerotic plaques. Scale bar, 100 μm. (FIG. 17C) Quantification of CD68+ macrophage load in plaques. (FIG. 17D) Quantification of the frequency of SR-A+ staining within macrophage population. Data are representative of two independent experiments, and error bars represent means±S.E.M. *P<0.05, **P<0.01 and ***P<0.001; Student's t-test (n=5 for each group).

FIGS. 18A-18B can demonstrate that superlow-dose LPS and oxLDL treatment elevates ROS accumulation in neutrophils. Neutrophils were purified from the bone marrow of wild-type C57 BL/6 mice and treated with PBS, super-low dose LPS (100 pg/ml) and/or oxLDL (10 μg/ml) for 2 d. (FIG. 18A) Representative histogram of ROS level determined by Cell ROX labeling. (FIG. 18B) Quantification of ROS level in neutrophils. Data are representative of three independent experiments, and error bars represent means±S.E.M. ***P<0.001; ANOVA (n=5 for each group).

FIGS. 19A-19B can demonstrate that 4-PBA reverses superlow-dose LPS-induced differential regulation of miR-24 and miR-126 in neutrophils. Neutrophils were purified from the bone marrow of wild-type C57 BL/6 mice and treated with PBS, super-low dose LPS (100 pg/ml) and/or 4-PBA (1 mM) for 2 d. The levels of miR-24 (FIG. 19A) and miR-126 (FIG. 19B) were determined by real-time RT-PCR. Data are representative of three independent experiments, and error bars represent means±S.E.M. *P<0.05, **P<0.01 and ***P<0.001; one-way ANOVA (n=4 for each group).

FIGS. 20A-20D can demonstrate that transfusion of 4-PBA-polarized neutrophils down-regulates plasma lipid levels and reduces lesional macrophage activation. Neutrophils purified from ApoE−/− mice were treated with PBS or 4-PBA (1 mM) for 24 h. PBS- or 4-PBA-treated neutrophils (2×106 cells per mouse) were then adoptively transferred by intravenous injection to HFD-fed ApoE−/− mice once a week for 4 weeks. (FIG. 20A) Plasma samples were collected 1 week after the last neutrophil transfer, and the levels of total cholesterol, free cholesterol and triglycerides were determined. (FIG. 20B) Representative confocal images of CD68 and SR-A staining in atherosclerotic plaques. Scale bar, 100 μm. (FIG. 20C) Quantification of CD68+ macrophage load in plaques. (FIG. 20D) Quantification of the frequency of SR-A+ staining within macrophage population. Data are representative of two independent experiments, and error bars represent means±S.E.M. *P<0.05, **P<0.01 and ***P<0.001; Student's t-test (n=7 for each group).

### DETAILED DESCRIPTION OF THE EXAMPLE EMBODIMENTS

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Where a range is expressed, a further aspect includes from the one particular value and/or to the other particular value. Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’”, where ‘x’ and ‘y’ are numerical values, includes “about ‘x’ to about ‘y’”.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used in the specification and the appended claims, the singular forms “a,” “an,” and “the” include plural referents unless the context clearly dictates otherwise.

The term “optional” or “optionally” means that the subsequent described event, circumstance or substituent may or may not occur, and that the description includes instances where the event or circumstance occurs and instances where it does not.

As used herein, “about,” “approximately,” “substantially,” and the like, when used in connection with a numerical variable, can generally refers to the value of the variable and to all values of the variable that are within the experimental error (e.g., within the 95% confidence interval for the mean) or within +/−10% of the indicated value, whichever is greater. As used herein, the terms “about,” “approximate,” “at or about,” and “substantially” can mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

Embodiments of the present disclosure will employ, unless otherwise indicated, techniques of molecular biology, microbiology, organic chemistry, biochemistry, physiology, cell biology, immunology, and the like, which are within the skill of the art. Such techniques are explained fully in the literature.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible unless the context clearly dictates otherwise.

### Definitions

Definitions of common terms and techniques in molecular biology may be found in Molecular Cloning: A Laboratory Manual, 2_{nd} edition (1989) (Sambrook, Fritsch, and Maniatis); Molecular Cloning: A Laboratory Manual, 4ₜₕ edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2_{nd} edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2_{nd} edition (2011).

As used herein, “active agent” or “active ingredient” can refer to a substance, compound, or molecule, which is biologically active or otherwise, induces a biological or physiological effect on a subject to which it is administered to. In other words, “active agent” or “active ingredient” refers to a component or components of a composition to which the whole or part of the effect of the composition is attributed.

As used herein, “administering” can refer to an administration that is oral, topical, intravenous, subcutaneous, transcutaneous, transdermal, intramuscular, intra joint, parenteral, intra-arteriole, intradermal, intraventricular, intraosseous, intraocular, intracranial, intraperitoneal, intralesional, intranasal, intracardiac, intraarticular, intracavernous, intrathecal, intravireal, intracerebral, and intracerebroventricular, intratympanic, intracochlear, rectal, vaginal, by inhalation, by catheters, stents or via an implanted reservoir or other device that administers, either actively or passively (e.g. by diffusion) a composition the perivascular space and adventitia. For example a medical device such as a stent can contain a composition or formulation disposed on its surface, which can then dissolve or be otherwise distributed to the surrounding tissue and cells. The term “parenteral” can include subcutaneous, intravenous, intramuscular, intra-articular, intra-synovial, intrasternal, intrathecal, intrahepatic, intralesional, and intracranial injections or infusion techniques.

As used herein, “agent” can refer to any substance, compound, molecule, and the like, which can be biologically active or otherwise can induce a biological and/or physiological effect on a subject to which it is administered to. An agent can be a primary active agent, or in other words, the component(s) of a composition to which the whole or part of the effect of the composition is attributed. An agent can be a secondary agent, or in other words, the component(s) of a composition to which an additional part and/or other effect of the composition is attributed.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

As used herein, “control” can refer to an alternative subject or sample used in an experiment for comparison purpose and included to minimize or distinguish the effect of variables other than an independent variable. A “suitable control” is one that will be instantly appreciated by one of ordinary skill in the art as one that is included such that it can be determined if the variable being evaluated an effect, such as a desired effect or hypothesized effect. One of ordinary skill in the art will also instantly appreciate based on inter alia, the context, the variable(s), the desired or hypothesized effect, what is a suitable or an appropriate control needed.

As used herein with reference to the relationship between DNA, cDNA, cRNA, RNA, protein/peptides, and the like “corresponding to” refers to the underlying biological relationship between these different molecules. As such, one of skill in the art would understand that operatively “corresponding to” can direct them to determine the possible underlying and/or resulting sequences of other molecules given the sequence of any other molecule which has a similar biological relationship with these molecules. For example, from a DNA sequence an RNA sequence can be determined and from an RNA sequence a cDNA sequence can be determined.

As used herein, “deoxyribonucleic acid (DNA)” and “ribonucleic acid (RNA)” generally refers to any polyribonucleotide or polydeoxyribonucleotide, which may be unmodified RNA or DNA or modified RNA or DNA. RNA can be in the form of non-coding RNA such as tRNA (transfer RNA), snRNA (small nuclear RNA), rRNA (ribosomal RNA), anti-sense RNA, RNAi (RNA interference construct), siRNA (short interfering RNA), microRNA (miRNA), or ribozymes, aptamers, guide RNA (gRNA) or coding mRNA (messenger RNA).

As used herein, “differentially expressed,” refers to the differential production of RNA, including but not limited to mRNA, tRNA, miRNA, siRNA, snRNA, and piRNA transcribed from a gene or regulatory region of a genome or the protein product encoded by a gene as compared to the level of production of RNA or protein by the same gene or regulator region in a normal or a control cell. In another context, “differentially expressed,” also refers to nucleotide sequences or proteins in a cell or tissue which have different temporal and/or spatial expression profiles as compared to a normal or control cell.

As used herein, “DNA molecule” can include nucleic acids/polynucleotides that are made of DNA.

As used herein, “effective amount” refers to the amount of a compound provided herein that is sufficient to effect beneficial or desired biological, emotional, medical, or clinical response of a cell, tissue, system, animal, or human. An effective amount can be administered in one or more administrations, applications, or dosages. The term cam also include within its scope amounts effective to enhance or restore to substantially normal physiological function. The “effective amount” can refer to the amount of a modified neutrophil as described herein that can be effective to reduce atherosclerosis, arteriosclerosis, myocardial infarction and injury, stroke, brain damage and trauma, traumatic brain injuries, multi-organ failure/sepsis, neuro-degenerative diseases (Parkinson's, Alzheimer's), or a related symptom thereof. Tools and/or approaches to generate neutrophils as described herein bearing similar homeostatic functional/phenotypic features described in this patent will include culturing neutrophils ex vivo with media supplemented with 4-PBA, phenylbutyrate, butyrate, and/or any other chemically-related derivatives. Genetic approaches such as introducing expression vectors to enhance the expression of Tollip, to reduce the expression of TICAM-2, or other genetic manipulations of neutrophils ex vivo to achieve the similar functional phenotype described herein will be covered by this patent. Approaches also include sorting/purifying autologous, homologous, or heterologous neutrophils bearing similar homeostatic functional/phenotypic features described in this patent to treat patients with related diseases.

As used herein, the term “encode” can refer to principle that DNA can be transcribed into RNA, which can then be translated into amino acid sequences that can form proteins.

As used herein, “expression” can refer to the process by which polynucleotides are transcribed into RNA transcripts. In the context of mRNA and other translated RNA species, “expression” also refers to the process or processes by which the transcribed RNA is subsequently translated into peptides, polypeptides, or proteins. In some instances, “expression” can also be a reflection of the stability of a given RNA. For example, when one measures RNA, depending on the method of detection and/or quantification of the RNA as well as other techniques used in conjunction with RNA detection and/or quantification, it can be that increased/decreased RNA transcript levels are the result of increased/decreased transcription and/or increased/decreased stability and/or degradation of the RNA transcript. One of ordinary skill in the art will appreciate these techniques and the relation “expression” in these various contexts to the underlying biological mechanisms.

As used herein, “gene” can refer to a hereditary unit corresponding to a sequence of DNA that occupies a specific location on a chromosome and that contains the genetic instruction for a characteristic(s) or trait(s) in an organism. The term gene can refer to translated and/or untranslated regions of a genome. “Gene” can refer to the specific sequence of DNA that is transcribed into an RNA transcript that can be translated into a polypeptide or be a catalytic RNA molecule, including but not limited to, tRNA, siRNA, piRNA, miRNA, long-non-coding RNA and shRNA.

As used herein, the terms “guide polynucleotide,” “guide sequence,” or “guide RNA” as can refer to any polynucleotide sequence having sufficient complementarity with a target polynucleotide sequence to hybridize with the target sequence and direct sequence-specific binding of a CRISPR complex to the target sequence. The degree of complementarity between a guide polynucleotide and its corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting examples of which include the Smith-Waterman algorithm, the Needleman-Wunsch algorithm, algorithms based on the Burrows-Wheeler Transform (e.g. the Burrows Wheeler Aligner), ClustalW, Clustal X, BLAT, Novoalign (Novocraft Technologies, ELAND (Illumina, San Diego, Calif.), SOAP (available at soap.genomics.org.cn), and Maq (available at maq.sourceforge.net). A guide polynucleotide (also referred to herein as a guide sequence and includes single guide sequences (sgRNA)) can be about or more than about 5, 10, 11, 12, 13, 14, 15, 16, 17, 18, 19, 20, 21, 22, 23, 24, 25, 26, 27, 28, 29, 30, 35, 40, 45, 50, 75, 90, 100, 110, 112, 115, 120, 130, 140, or more nucleotides in length. The guide polynucleotide can include a nucleotide sequence that is complementary to a target DNA sequence. This portion of the guide sequence can be referred to as the complementary region of the guide RNA. In some contexts, the two are distinguished from one another by calling one the complementary region or target region and the rest of the polynucleotide the guide sequence or tracrRNA. The guide sequence can also include one or more miRNA target sequences coupled to the 3′ end of the guide sequence. The guide sequence can include one or more MS2 RNA aptamers incorporated within the portion of the guide strand that is not the complementary portion. As used herein the term guide sequence can include any specially modified guide sequences, including but not limited to those configured for use in synergistic activation mediator (SAM) implemented CRISPR (Nature 517, 583-588 (29 Jan. 2015) or suppression (Cell Volume 154, Issue 2, 18 Jul. 2013, Pages 442-451). A guide polynucleotide can be less than about 150, 125, 75, 50, 45, 40, 35, 30, 25, 20, 15, 12, or fewer nucleotides in length. The ability of a guide polynucleotide to direct sequence-specific binding of a CRISPR complex to a target sequence may be assessed by any suitable assay. For example, the components of a CRISPR system sufficient to form a CRISPR complex, including the guide polynucleotide to be tested, may be provided to a host cell having the corresponding target sequence, such as by transfection with vectors encoding the components of the CRISPR sequence, followed by an assessment of preferential cleavage within the target sequence. Similarly, cleavage of a target polynucleotide sequence may be evaluated in a test tube by providing the target sequence, components of a CRISPR complex, including the guide polynucleotide to be tested and a control guide polynucleotide different from the test guide polynucleotide, and comparing binding or rate of cleavage at the target sequence between the test and control guide polynucleotide reactions. Other assays are possible, and will occur to those skilled in the art.

A complementary region of the gRNA can be configured to target any DNA region of interest. The complementary region of the gRNA and the gRNA can be designed using a suitable gRNA design tool. Suitable tools are known in the art and are available to the skilled artisan. As such, the constructs described herein are enabled for any desired target DNA so long as it is CRISPR compatible according to the known requirements for CRISPR activation.

A guide polynucleotide can be selected to reduce the degree of secondary structure within the guide polynucleotide. Secondary structure may be determined by any suitable polynucleotide folding algorithm. Some programs are based on calculating the minimal Gibbs free energy. An example of one such algorithm is mFold, as described by Zuker & Stiegler ((1981) Nucleic Acids Res. 9, 133-148). Another example folding algorithm is the online webserver RNAfold, developed at Institute for Theoretical Chemistry at the University of Vienna, using the centroid structure prediction algorithm (see e.g. Gruber et al., (2008) Cell 106: 23-24; and Carr & Church (2009) Nature Biotechnol. 27: 1151-1162).

As used herein, the term “homology-directed repair (HDR)” can refer to a mechanism in cells to repair double-stranded and single stranded DNA breaks. Homology-directed repair includes homologous recombination (HR) and single-strand annealing (SSA) (Lieber. (2010) Annu. Rev. Biochem. 79: 181-211). The most common form of HDR is called homologous recombination (HR), which has the longest sequence homology requirements between the donor and acceptor DNA. Other forms of HDR include single-stranded annealing (SSA) and breakage-induced replication, and these require shorter sequence homology relative to HR. Homology-directed repair at nicks (single-stranded breaks) can occur via a mechanism distinct from HDR at double-strand breaks.

As used herein, “identity,” can refer to a relationship between two or more nucleotide or polypeptide sequences, as determined by comparing the sequences. In the art, “identity” can also refer to the degree of sequence relatedness between nucleotide or polypeptide sequences as determined by the match between strings of such sequences. “Identity” can be readily calculated by known methods, including, but not limited to, those described in (Computational Molecular Biology, Lesk, A. M., Ed., Oxford University Press, New York, 1988; Biocomputing: Informatics and Genome Projects, Smith, D. W., Ed., Academic Press, New York, 1993; Computer Analysis of Sequence Data, Part I, Griffin, A. M, and Griffin, H G., Eds., Humana Press, New Jersey, 1994; Sequence Analysis in Molecular Biology, von Heinje, G., Academic Press, 1987; and Sequence Analysis Primer, Gribskov, M and Devereux, J., Eds., M Stockton Press, New York, 1991; and Carillo, H, and Lipman, D., SIAM J. Applied Math. 1988, 48: 1073. Preferred methods to determine identity are designed to give the largest match between the sequences tested. Methods to determine identity are codified in publicly available computer programs. The percent identity between two sequences can be determined by using analysis software (e.g., Sequence Analysis Software Package of the Genetics Computer Group, Madison Wis.) that incorporates the Needelman and Wunsch, (J. Mol. Biol., 1970, 48: 443-453,) algorithm (e.g., NBLAST, and XBLAST). The default parameters are used to determine the identity for the polypeptides of the present disclosure, unless stated otherwise.

As used herein, “microRNA” refers to a small non-coding RNA molecule containing about 21 to about 23 nucleotides found in organisms, which functions in transcriptional and post-transcriptional regulation of transcription and translation of RNA. “MicroRNA” can exist as part of a larger nucleic acid molecule such as a stem-loop structure that can be processed by a cell and yield a microRNA of about 21-23 nucleotides.

The term “molecular weight”, as used herein, generally refers to the mass or average mass of a material. If a polymer or oligomer, the molecular weight can refer to the relative average chain length or relative chain mass of the bulk polymer. In practice, the molecular weight of polymers and oligomers can be estimated or characterized in various ways including gel permeation chromatography (GPC) or capillary viscometry. GPC molecular weights are reported as the weight-average molecular weight (M_{w}) as opposed to the number-average molecular weight (Mₙ). Capillary viscometry provides estimates of molecular weight as the inherent viscosity determined from a dilute polymer solution using a particular set of concentration, temperature, and solvent conditions.

As used herein, “negative control” refers to a “control” that is designed to produce no effect or result, provided that all reagents are functioning properly and that the experiment is properly conducted. Other terms that are interchangeable with “negative control” include “sham,” “placebo,” and “mock.”

As used herein, “nucleic acid,” “nucleotide sequence,” and “polynucleotide” can be used interchangeably herein and generally refers to a string of at least two base-sugar-phosphate combinations and refers to, among others, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, polynucleotide as used herein can refer to triple-stranded regions comprising RNA or DNA or both RNA and DNA. The strands in such regions can be from the same molecule or from different molecules. The regions may include all of one or more of the molecules, but more typically involve only a region of some of the molecules. One of the molecules of a triple-helical region often is an oligonucleotide. “Polynucleotide” and “nucleic acids” also encompasses such chemically, enzymatically or metabolically modified forms of polynucleotides, as well as the chemical forms of DNA and RNA characteristic of viruses and cells, including simple and complex cells, inter alia. For instance, the term polynucleotide as used herein can include DNAs or RNAs as described herein that contain one or more modified bases. Thus, DNAs or RNAs including unusual bases, such as inosine, or modified bases, such as tritylated bases, to name just two examples, are polynucleotides as the term is used herein. “Polynucleotide”, “nucleotide sequences” and “nucleic acids” also includes PNAs (peptide nucleic acids), phosphorothioates, and other variants of the phosphate backbone of native nucleic acids. Natural nucleic acids have a phosphate backbone, artificial nucleic acids can contain other types of backbones, but contain the same bases. Thus, DNAs or RNAs with backbones modified for stability or for other reasons are “nucleic acids” or “polynucleotides” as that term is intended herein. As used herein, “nucleic acid sequence” and “oligonucleotide” also encompasses a nucleic acid and polynucleotide as defined elsewhere herein.

As used herein, “operatively linked” in the context of recombinant DNA molecules, vectors, and the like refers to the regulatory and other sequences useful for expression, stabilization, replication, and the like of the coding and transcribed non-coding sequences of a nucleic acid that are placed in the nucleic acid molecule in the appropriate positions relative to the coding sequence so as to effect expression or other characteristic of the coding sequence or transcribed non-coding sequence. This same term can be applied to the arrangement of coding sequences, non-coding and/or transcription control elements (e.g. promoters, enhancers, and termination elements), and/or selectable markers in an expression vector. “Operatively linked” can also refer to an indirect attachment (i.e. not a direct fusion) of two or more polynucleotide sequences or polypeptides to each other via a linking molecule (also referred to herein as a linker).

As used herein, “overexpressed” or “overexpression” refers to an increased expression level of an RNA and/or protein product encoded by a gene as compared to the level of expression of the RNA or protein product in a normal or control cell. The amount of increased expression as compared to a normal or control cell can be about 0.1, 0.2, 0.3, 0.4, 0.5, 0.6, 0.7, 0.8, 0.9, 1.0, 1.2, 1.3, 1.4, 1.5, 1.6, 1.7, 1.8, 1.9, 2.0, 2.2, 2.4, 2.6, 2.8, 3.0, 3.3, 3.6, 3.9, 4.0, 4.4, 4.8, 5.0, 5.5, 6, 6.5, 7, 7.5, 8.0, 8.5, 9, 9.5, 10, 11, 12, 13, 14, 15, 16, 17, 18, 19, 20, 25, 30, 35, 40, 45, 50, 60, 70, 0, 90, 100 fold or more greater than the normal or control cell.

As used herein, “patient” refers to an organism, host, or subject in need of treatment.

As used herein “peptide” refers to chains of at least 2 amino acids that are short, relative to a protein or polypeptide.

As used herein, “pharmaceutical formulation” refers to the combination of an active agent, compound, or ingredient with a pharmaceutically acceptable carrier or excipient, making the composition suitable for diagnostic, therapeutic, or preventive use in vitro, in vivo, or ex vivo.

As used herein, “pharmaceutically acceptable carrier or excipient” refers to a carrier or excipient that is useful in preparing a pharmaceutical formulation that is generally safe, non-toxic, and is neither biologically or otherwise undesirable, and includes a carrier or excipient that is acceptable for veterinary use as well as human pharmaceutical use. A “pharmaceutically acceptable carrier or excipient” as used in the specification and claims includes both one and more than one such carrier or excipient.

As used herein, “pharmaceutically acceptable salt” refers to any acid or base addition salt whose counter-ions are non-toxic to the subject to which they are administered in pharmaceutical doses of the salts.

As used herein, “plasmid” refers to a non-chromosomal double-stranded DNA sequence including an intact “replicon” such that the plasmid is replicated in a host cell.

As used herein, “positive control” refers to a “control” that is designed to produce the desired result, provided that all reagents are functioning properly and that the experiment is properly conducted.

As used herein, “preventative” and “prevent” refers to hindering or stopping a disease or condition before it occurs, even if undiagnosed, or while the disease or condition is still in the sub-clinical phase.

As used herein, “polypeptides” or “proteins” refers to amino acid residue sequences. Those sequences are written left to right in the direction from the amino to the carboxy terminus. In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V). “Protein” and “Polypeptide” can refer to a molecule composed of one or more chains of amino acids in a specific order. The term protein is used interchangeable with “polypeptide.” The order is determined by the base sequence of nucleotides in the gene coding for the protein. Proteins can be required for the structure, function, and regulation of the body's cells, tissues, and organs.

As used herein, “promoter” includes all sequences capable of driving transcription of a coding or a non-coding sequence. In particular, the term “promoter” as used herein refers to a DNA sequence generally described as the 5′ regulator region of a gene, located proximal to the start codon. The transcription of an adjacent coding sequence(s) is initiated at the promoter region. The term “promoter” also includes fragments of a promoter that are functional in initiating transcription of the gene.

As used herein, the term “recombinant” or “engineered” can generally refer to a non-naturally occurring nucleic acid, nucleic acid construct, or polypeptide. Such non-naturally occurring nucleic acids may include natural nucleic acids that have been modified, for example that have deletions, substitutions, inversions, insertions, etc., and/or combinations of nucleic acid sequences of different origin that are joined using molecular biology technologies (e.g., a nucleic acid sequences encoding a fusion protein (e.g., a protein or polypeptide formed from the combination of two different proteins or protein fragments), the combination of a nucleic acid encoding a polypeptide to a promoter sequence, where the coding sequence and promoter sequence are from different sources or otherwise do not typically occur together naturally (e.g., a nucleic acid and a constitutive promoter), etc. Recombinant or engineered can also refer to the polypeptide encoded by the recombinant nucleic acid. Non-naturally occurring nucleic acids or polypeptides include nucleic acids and polypeptides modified by man.

As used herein, “seed sequence” or “seed region” refers to a 7 nucleotide long region within a microRNA that can be conserved between 2 or more microRNAs that is typically located from nucleotides 2-7 from the 5′ end of the mature microRNA.

As used herein, the term “specific binding” can refer to non-covalent physical association of a first and a second moiety wherein the association between the first and second moieties is at least 2 times as strong, at least 5 times as strong as, at least 10 times as strong as, at least 50 times as strong as, at least 100 times as strong as, or stronger than the association of either moiety with most or all other moieties present in the environment in which binding occurs. Binding of two or more entities may be considered specific if the equilibrium dissociation constant, Kd, is 10₋₃ M or less, 10₋₄ M or less, 10₋₅ M or less, 10₋₆ M or less, 10₋₇ M or less, 10₋₈ M or less, 10₋₉ M or less, 10₋₁₀ M or less, 10₋₁₁ M or less, or 10₋₁₂ M or less under the conditions employed, e.g., under physiological conditions such as those inside a cell or consistent with cell survival. In some embodiments, specific binding can be accomplished by a plurality of weaker interactions (e.g., a plurality of individual interactions, wherein each individual interaction is characterized by a Kd of greater than 10₋₃ M). In some embodiments, specific binding, which can be referred to as “molecular recognition,” is a saturable binding interaction between two entities that is dependent on complementary orientation of functional groups on each entity. Examples of specific binding interactions include primer-polynucleotide interaction, aptamer-aptamer target interactions, antibody-antigen interactions, avidin-biotin interactions, ligand-receptor interactions, metal-chelate interactions, hybridization between complementary nucleic acids, etc.

As used interchangeably herein, “subject,” “individual,” or “patient” can refer to a vertebrate organism, such as a mammal (e.g. human). “Subject” can also refer to a cell, a population of cells, a tissue, an organ, or an organism, preferably to human and constituents thereof.

As used herein, “substantially pure” can mean an object species is the predominant species present (i.e., on a molar basis it is more abundant than any other individual species in the composition), and preferably a substantially purified fraction is a composition wherein the object species comprises about 50 percent of all species present. Generally, a substantially pure composition will comprise more than about 80 percent of all species present in the composition, more preferably more than about 85%, 90%, 95%, and 99%. Most preferably, the object species is purified to essential homogeneity (contaminant species cannot be detected in the composition by conventional detection methods) wherein the composition consists essentially of a single species.

As used interchangeably herein, the terms “sufficient” and “effective,” can refer to an amount (e.g. mass, volume, dosage, concentration, and/or time period) needed to achieve one or more desired result(s). For example, a therapeutically effective amount refers to an amount needed to achieve one or more therapeutic effects.

As used herein, “therapeutic” can refer to treating, healing, and/or ameliorating a disease, disorder, condition, or side effect, or to decreasing in the rate of advancement of a disease, disorder, condition, or side effect. A “therapeutically effective amount” can therefore refer to an amount of a compound that can yield a therapeutic effect. The therapeutic effect can be treating and/or preventing non-resolving inflammation and/or related diseases or conditions, including but not limited to atherosclerosis, cardiovascular disease, stroke, myocardial infarction, a neurological disease, and/or a symptom thereof in the subject in need thereof. Neurological diseases in this context can include, but are not limited to Parkinson's disease, Alzheimer's disease, neurotrophic viral disease, and paraneoplastic disorders, and other neurodegenerative diseases. The therapeutic effect can be decreasing circulating amount of miR-24 in a subject, increasing the circulating amount of miR-126 in a subject, decrease plaque size in a subject, decrease plasma protein levels of MPO, LTB4, and/or MMP9 in a subject, increase plasma levels of TGFβ in a subject, increase levels of plaque collagen in a subject, increase surface levels of CD62L in one or more neutrophils in a subject, decrease gene and/or protein expression levels of CD11b and/or Dectin-1 in one or more neutrophils in a subject, increase gene and/or protein expression levels of FPN, LRRC32 in one or more neutrophils in a subject, or any combination thereof. The ex vivo programmed autologous or homologous neutrophils with 4-PBA and/or its chemical derivatives, or with genetic approaches can be used to treat atherosclerosis, arteriosclerosis, myocardial infarction and injury, stroke, brain damage and trauma, traumatic brain injuries, multi-organ failure/sepsis, neuro-degenerative diseases (Parkinson's, Alzheimer's), or a related symptom thereof.

As used herein, the terms “treating” and “treatment” can refer generally to obtaining a desired pharmacological and/or physiological effect. The effect can be, but does not necessarily have to be, prophylactic in terms of preventing or partially preventing a disease, symptom or condition thereof, including, but not limited to, non-resolving inflammation and/or related diseases or conditions, including but not limited to atherosclerosis, cardiovascular disease, stroke, myocardial infarction, a neurological disease, and/or a symptom thereof in the subject in need thereof. The effect can be therapeutic in terms of a partial or complete cure of a disease, condition, symptom or adverse effect attributed to the disease, disorder, or condition. The term “treatment” as used herein covers any treatment of cancer, in a subject, particularly a human, and can include any one or more of the following: (a) preventing the disease from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, such as treating the pain of a subject by administration of an analgesic agent even though such agent does not treat the cause of the pain.

As used herein, “underexpressed” or “underexpression” can refer to decreased expression level of an RNA (coding or non-coding RNA) or protein product encoded by a gene as compared to the level of expression of the RNA or protein product in a normal or control cell. The amount of decreased expression as compared to a normal or control cell can be about 0.1, 0.2, 0.3, 0.4, 0.5, 0.6, 0.7, 0.8, 0.9, 1.0, 1.2, 1.3, 1.4, 1.5, 1.6, 1.7, 1.8, 1.9, 2.0, 2.2, 2.4, 2.6, 2.8, 3.0, 3.3, 3.6, 3.9, 4.0, 4.4, 4.8, 5.0, 5.5, 6, 6.5, 7, 7.5, 8.0, 8.5, 9, 9.5, 10, 11, 12, 13, 14, 15, 16, 17, 18, 19, 20, 25, 30, 35, 40, 45, 50, 60, 70, 0, 90, 100 fold or more less than the normal or control cell.

As used herein, “variant” can refer to a polynucleotide or polypeptide that differs from a reference polynucleotide or polypeptide, but retains essential and/or characteristic properties (structural and/or functional) of the reference polynucleotide or polypeptide. A typical variant of a polypeptide differs in amino acid sequence from another, reference polypeptide. The differences can be limited so that the sequences of the reference polypeptide and the variant are closely similar overall and, in many regions, identical. A variant and reference polypeptide may differ in nucleic or amino acid sequence by one or more modifications at the sequence level or post-transcriptional or post-translational modifications (e.g., substitutions, additions, deletions, methylation, glycosylations, etc.). A substituted nucleic acid may or may not be an unmodified nucleic acid of adenine, thiamine, guanine, cytosine, uracil, including any chemically, enzymatically or metabolically modified forms of these or other nucleotides. A substituted amino acid residue may or may not be one encoded by the genetic code. A variant of a polypeptide may be naturally occurring such as an allelic variant, or it may be a variant that is not known to occur naturally. “Variant” includes functional and structural variants.

As used herein, the term “vector” or is used in reference to a vehicle used to introduce an exogenous nucleic acid sequence into a cell. A vector may include a DNA molecule, linear or circular (e.g. plasmids), which includes a segment encoding a polypeptide of interest operatively linked to additional segments that provide for its transcription and translation upon introduction into a host cell or host cell organelles. Such additional segments may include promoter and terminator sequences, and may also include one or more origins of replication, one or more selectable markers, an enhancer, a polyadenylation signal, etc. Expression vectors are generally derived from yeast or bacterial genomic or plasmid DNA, or viral DNA, or may contain elements of both. Suitable vectors, including expression vectors, are generally known in the art and will be appreciated by one of ordinary skill in the art in view of this disclosure.

As used herein, “wild-type” refers to the typical or average from of a gene, protein, species, organism, etc. as it occurs in a given population.

As used herein, “transforming” when used in the context of engineering or modifying a cell, refers to the introduction by any suitable technique and/or the transient or stable incorporation and/or expression of an exogenous gene in a cell.

As used herein, “suicide gene” refers to a gene that encodes one or more proteins that can result in apoptosis of that cell and can be inducible upon administration or contact with an exogenous molecule or agent so as to provide exogenously controlled apoptosis of a cell that carries one or more suicide genes. These can also be referred to as “elimination genes”. A variety of suicide genes can be employed for this purpose, including HSV-TK (herpes simplex virus thymidine kinase), Fas, iCasp9 (inducible caspase 9), CD20, MYC TAG, and truncated EGFR (endothelial growth factor receptor). HSK for example, will convert the prodrug ganciclovir (GCV) into GCV-triphosphate that incorporates itself into replicating DNA, ultimately leading to cell death. iCasp9 is a chimeric protein containing components of FK506-binding protein that binds the small molecule AP1903, leading to caspase 9 dimerization and apoptosis. Other suitable suicide genes will be appreciated by those of ordinary skill in the art. Suicide genes can function to provide a route to specifically remove modified cells from a subject.

### Overview

Atherosclerosis and related cardiovascular and vascular complications are leading causes of morbidity and mortality world-wide with serious economic and heath tolls. A key risk factor for atherosclerosis is the establishment of non-resolving inflammation. However, the limited understanding of the underlying mechanisms of the establishment of non-resolving inflammation is a major road-block for the development of effective prevention and treatments. As such there exists a need for compositions and techniques for understanding, treating, and/or preventing non-resolving inflammation and diseases, such as atherosclerosis and related cardiovascular and vascular diseases and conditions.

With that said, described herein are chemically programmed neutrophils that can be used to treat and/or prevent non-resolving inflammation and diseases, including, but not limited to, atherosclerosis and related cardiovascular and vascular diseases and conditions. They can also be used to treat neurological diseases with a non-resolving inflammatory component, including but not limited to Parkinson's disease, Alzheimer's disease, neurotropic viral infections, paraneoplastic disorders, and other neurodegenerative diseases. Other compositions, compounds, methods, features, and advantages of the present disclosure will be or become apparent to one having ordinary skill in the art upon examination of the following drawings, detailed description, and examples. It is intended that all such additional compositions, compounds, methods, features, and advantages be included within this description, and be within the scope of the present disclosure.

Chemically Reprogrammed Neutrophils and Pharmaceutical Formulations Thereof

Neutrophils constitute about 50-70% of circulating white blood cells and have been observed to be elevated in circulating blood as well as atherosclerotic plaques from human and animals having unstable plaques. Neutrophils can be differentially polarized and can both promote or inhibit inflammatory states depending on the state of the neutrophil. The chemically re-programmed neutrophils described herein are manipulated to be in a state that can inhibit the inflammatory state and be used as a treatment and/or prevention for non-resolving inflammation and related diseases.

Described herein are neutrophils that have been programmed with 4-phenylbutyrate (4-PBA) or a pharmaceutically acceptable salt thereof. The chemically programmed neutrophils described herein can have increased gene and/or protein expression levels of LLRC32, TGFβ, CD62L and/or FPN as compared to neutrophils having a non-resolving inflammation phenotype. The chemically programmed neutrophils described herein can have decreased gene and/or protein expression levels of CD11b, MMP9, MPO, LTB4, and/or Dectin-1 as compared to a neutrophil having a non-resolving inflammation phenotype.

A non-resolving inflammation phenotype in neutrophils can be characterized by increased levels of gene and/or protein expression of the inflammatory mediators Dectin-1, MMP9 and LTB4 and decreased levels of gene and/or protein expression of the homeostatic mediators LRRC32, TGFβ, and FPN as compared to a normal or other suitable control. Further, neutrophils with a non-resolving inflammation phenotype can have increased activation of oxCAMKII as compared to a normal or other suitable control, which is caused by altered peroxisome homeostasis and reduced lysosome function.

The chemically programmed neutrophils can be generated by contacting a neutrophil or a population thereof with an amount of 4-PBA. In some embodiments, the neutrophil can have a non-resolving inflammation phenotype. In some embodiments, the neutrophil does not have non-resolving inflammation phenotype. In some embodiments, the neutrophil can be harvested from a subject and the step of contacting the neutrophil with an amount of 4-PBA can occur ex vivo. In some embodiments, the neutrophil harvested from the subject can be cultured and/or otherwise manipulated (e.g. to expand, proliferate, store, genetically modify, etc.) in addition to being contacted with the amount of 4-PBA). Suitable culture reagents, methods, and techniques for culturing, expanding, and genetically manipulating neutrophils will be instantly appreciated by those of ordinary skill in the art. In some embodiments, the neutrophils harvested from the subject are genetically modified to express a suicide gene or marker to allow identification and/or destruction of the chemically programmed neutrophils. The concentration of 4-PBA or related butyrate derivatives that the neutrophils are contacted with can range from about 0.001, 0.01, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.5, 6.0, 6.5, 7.0, 7.5, 8.0, 8.5, 9.0. 10, 11, 12, 13, 14, 15, 16, 17, 18, 19, 20, 30, 40, 50, 75, 100, 250, 500, 750, to about 1000 mM or more. The amount of time that the neutrophils are kept in contact with the 4-PBA can range from about 1 hour to 6 h, 12 h, 18 h, 24 h, 36 h, 48 h, 72 h, or more.

Also described herein are formulations, such as pharmaceutical formulations, that can include a chemically programmed neutrophil as described herein or a population thereof and a carrier, such as a pharmaceutically acceptable carrier. The formulation, such as a pharmaceutical formulation, can include a therapeutically effective amount of the chemically programmed neutrophil or population thereof. The therapeutically effective amount can range from about 100 to 1×10₁, 1×10₂, 1×10₃, 1×10₄, 1×10₅, 1×10₆, 1×10₇, 1×10₈, 1×10₉, 1×10₁₀, 1×10₁₁, 1×10₁₂, 1×10₁₃, 1×10₁₄, 1×10₁₅, 1×10₁₆, 1×10₁₇, 1×10₁₈, 1×10₁₉, 1×10₂₀ or more cells or cells/mL. The pharmaceutical formulations can be used to treat and/or prevent non-resolving inflammation and/or related diseases or conditions, including but not limited to atherosclerosis, cardiovascular disease, stroke, myocardial infarction, a neurological disease, and/or a symptom thereof.

Suitable pharmaceutically acceptable carriers include, but are not limited to, water, salt solutions, alcohols, gum arabic, vegetable oils, benzyl alcohols, polyethylene glycols, gelatin, carbohydrates such as lactose, amylose or starch, magnesium stearate, talc, silicic acid, viscous paraffin, perfume oil, fatty acid esters, hydroxy methylcellulose, and polyvinyl pyrrolidone, which do not deleteriously react with the active composition.

The pharmaceutical formulations can be sterilized, and if desired, mixed with auxiliary agents, such s lubricants, preservatives, stabilizers, wetting agents, emulsifiers, salts for influencing osmotic pressure, buffers, coloring, flavoring and/or aromatic substances, and the like which do not deleteriously react with the active composition.

In addition to the therapeutically effective amount of the chemically programmed neutrophil r population thereof the pharmaceutical formulation can also include an effective amount of an auxiliary active agent, including but not limited to, DNA, RNA, amino acids, peptides, polypeptides, antibodies, aptamers, ribozymes, guide sequences for ribozymes that inhibit translation or transcription of essential tumor proteins and genes, hormones, immunomodulators, antipyretics, anxiolytics, antipsychotics, analgesics, antispasmodics, anti-inflammatories, anti-histamines, anti-infectives, chemotherapeutics and combinations thereof.

In embodiments where there is an auxiliary active agent contained in the pharmaceutical formulation in addition to the chemically programmed neutrophil, the therapeutically effective amount of the auxiliary active agent will vary depending on the auxiliary active agent. In some embodiments, the effective amount of the auxiliary active agent ranges from 0.001 micrograms to about 1 milligram, such as 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, or 1 micrograms or milligrams. In other embodiments, the effective amount of the auxiliary active agent ranges from about 0.01 IU to about 1000 IU, such as about 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or about 1000 IU. In further embodiments, the effective amount of the auxiliary active agent ranges from 0.001 mL to about 1 mL, such as about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, or about 1 mL. In yet other embodiments, the effective amount of the auxiliary active agent ranges from about 1% to about 50% w/w, v/v, or w/v, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50% w/w, v/v, or w/v of the total pharmaceutical formulation.

### Dosage Forms

In some embodiments, the pharmaceutical formulations described herein may be in a dosage form. The dosage forms can be adapted for administration by any appropriate route. The preferred route of administration is intravenous injection of programmed neutrophils. Other appropriate routes can include, but are not limited to epidural, intracranial, intraocular, vaginal, intraurethral, parenteral, intracranial, subcutaneous, intramuscular, intravenous, intraperitoneal, intradermal, intraosseous, intracardiac, intraarticular, intracavernous, intrathecal, intravitreal, intracerebral, gingival, subgingival, intracerebroventricular, and intradermal. In addition, compound administration with 4-PBA or its related butyrate derivatives may also include oral (including buccal or sublingual), rectal, inhaled, intranasal, and topical (including buccal, sublingual, or transdermal). Such formulations may be prepared by any method known in the art.

Dosage forms adapted for parenteral administration and/or adapted for any type of injection (e.g. intravenous, intraperitoneal, subcutaneous, intramuscular, intradermal, intraosseous, epidural, intracardiac, intraarticular, intracavernous, gingival, subgingival, intrathecal, intravireal, intracerebral, and intracerebroventricular) can include aqueous and/or non-aqueous sterile injection solutions, which can contain anti-oxidants, buffers, bacteriostats, solutes that render the composition isotonic with the blood of the subject, and aqueous and non-aqueous sterile suspensions, which can include suspending agents and thickening agents. The dosage forms adapted for parenteral administration can be presented in a single-unit dose or multi-unit dose containers, including but not limited to sealed ampoules or vials. The doses can be lyophilized and resuspended in a sterile carrier to reconstitute the dose prior to administration. Extemporaneous injection solutions and suspensions can be prepared in some embodiments, from concentrated cell solutions, sterile powders, granules, and tablets.

For some embodiments, the dosage form contains a predetermined amount of the chemically programmed neutrophils per unit dose. In some embodiments, the predetermined amount of the chemically programmed neutrophils is a therapeutically effective amount of the chemically programmed neutrophils, effective to treat or prevent non-resolving inflammation and/or related diseases or conditions, including but not limited to atherosclerosis, cardiovascular disease, stroke, myocardial infarction, neurological disease, and/or a symptom thereof. In other embodiments, the predetermined amount of the chemically programmed neutrophils can be an appropriate fraction of the therapeutically effective amount of the active ingredient (e.g. the chemically programmed neutrophils and/or auxiliary active agent). Such unit doses may therefore be administered once or more than once a day. Such pharmaceutical formulations may be prepared by any of the methods well known in the art.

### Methods of Using the Chemically Modified Neutrophils

The chemically programmed neutrophils and pharmaceutical formulations thereof described herein can be used for the treatment and/or prevention of a disease, disorder, syndrome, or a symptom thereof in a subject. In some embodiments, the chemically programmed and pharmaceutical formulations thereof described herein can be used to treat and/or prevent non-resolving inflammation and/or related diseases or conditions, including but not limited to atherosclerosis, cardiovascular disease, stroke, myocardial infarction, a neurological disease, and/or a symptom thereof in a subject.

An amount of the chemically programmed neutrophils and pharmaceutical formulations thereof described herein can be administered to a subject in need thereof one or more times per day, week, month, or year. In some embodiments, the amount administered can be the therapeutically effective amount of the chemically programmed neutrophils or pharmaceutical formulations thereof. For example, the chemically programmed neutrophils or pharmaceutical formulations thereof can be administered in a daily dose. This amount may be given in a single dose per day. In other embodiments, the daily dose may be administered over multiple doses per day, in which each containing a fraction of the total daily dose to be administered (sub-doses). In some embodiments, the amount of doses delivered per day is 2, 3, 4, 5, or 6. In further embodiments, the chemically programmed neutrophils and pharmaceutical formulations thereof can be administered one or more times per week, such as 1, 2, 3, 4, 5, or 6 times per week. In other embodiments, the chemically programmed neutrophils and pharmaceutical formulations thereof can be administered one or more times per month, such as 1 to 5 times per month, such as 1, 2, 3, 4 or 5 times per month. In still further embodiments, the chemically programmed neutrophils and pharmaceutical formulations thereof can be administered one or more times per year, such as 1 to 11 times per year. In some embodiments, the chemically programmed neutrophils and pharmaceutical formulations thereof can be administered 1, 2, 3, 4, 5, 6, 7, 8, 9, 10, 11 times per year.

The chemically programmed neutrophils and pharmaceutical formulations thereof can be co-administered with a secondary agent by any convenient route. The secondary agent is a separate compound and/or pharmaceutical formulation from the chemically programmed neutrophils or pharmaceutical formulations thereof. The secondary agent can be administered simultaneously with the chemically programmed neutrophils or pharmaceutical formulations thereof. The secondary agent can be administered sequentially with the chemically programmed neutrophils or pharmaceutical formulations thereof. Suitable secondary agents include, but are not limited to, DNA, RNA, amino acids, peptides, polypeptides, antibodies, aptamers, ribozymes, guide sequences for ribozymes that inhibit translation or transcription of essential tumor proteins and genes, hormones, immunomodulators, antipyretics, anxiolytics, antipsychotics, analgesics, antispasmodics, anti-inflammatories, anti-histamines, anti-infectives, and chemotherapeutics.

In embodiments where the chemically programmed neutrophils or pharmaceutical formulations thereof are simultaneously co-administered with a secondary agent, the chemically programmed neutrophils or pharmaceutical formulations thereof can be administered to the subject at substantially the same time as the secondary agent. As used in this context “substantially the same time” refers to administration of chemically programmed neutrophils or pharmaceutical formulations thereof and a secondary agent where the period of time between administration of the chemically programmed neutrophils or pharmaceutical formulations thereof and the secondary agent is between 0 and 10 minutes (e.g. 0 (simultaneous), 1, 2, 3, 4, 5, 6, 7, 8, 9, or 10 minutes).

In embodiments where the chemically programmed neutrophils or pharmaceutical formulations thereof is/are sequentially co-administered with a secondary agent, the chemically programmed neutrophils or pharmaceutical formulations thereof can be administered first, and followed by administration of the secondary agent after a period of time. In other embodiments where the chemically programmed neutrophils or pharmaceutical formulations thereof is/are sequentially co-administered with a secondary agent, the secondary agent can be administered first, and followed by administration of the chemically programmed neutrophils or pharmaceutical formulations thereof after a period of time. The period of time between administration of the chemically programmed neutrophils or pharmaceutical formulations thereof and the secondary agent can range from 10 minutes to about 96 hours, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, to/or 96 hours. In some embodiments the period of time can be about 10 minutes, about 30 minutes, about 1 hour, about 2 hours, about 4 hours, about 6 hours, about 8 hours, about 10 hours, or about 12 hours. The sequential administration can be repeated as necessary over the course of the period of treatment.

The amount of the chemically programmed neutrophils or pharmaceutical formulations thereof that can be administered are described elsewhere herein. The amount of the secondary agent will vary depending on the secondary agent. The amount of the secondary agent can be a therapeutically effective amount. In some embodiments, the effective amount of the secondary agent ranges from 0.001 micrograms to about 1 milligram, such as 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, or 1 micrograms or milligrams. In other embodiments, the amount of the secondary agent ranges from about 0.01 IU to about 1000 IU, such as about 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or about 1000 IU. In further embodiments, the amount of the secondary agent ranges from 0.001 mL to about 1 mL, such as about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, or about 1 mL. In yet other embodiments, the amount of the secondary agent ranges from about 1% to about 50%, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 w/w, v/v, or w/v of the total secondary agent composition or pharmaceutical formulation.

In some embodiments, the chemically programmed neutrophils or pharmaceutical formulations thereof can be administered to a patient via an injection. Suitable methods of injection include, but are not limited to, intravenous, intraperitoneal, subcutaneous, intramuscular, intradermal, intraosseous, epidural, intracardiac, intraarticular, intracavernous, intrathecal, intravitreal, intracerebral, gingival, subgingival, intranodal, and intracerebroventricular injection. Other suitable methods of administration of the composition or formulation containing the chemically programmed neutrophils or pharmaceutical formulations thereof can include, but are not limited to, subcutaneous, intravenous, and/or parenteral. In some embodiments, the preferred administration route is intravenous. In some embodiments, the dosage of the chemically programmed neutrophils or pharmaceutical formulation thereof ranges from about 0.01 million neutrophils/kg to 1 million neutrophils/kg, or from 0.01 μg pharmaceutical formulation/kg bodyweight to about 1 mg pharmaceutical formulation/kg bodyweight. In some embodiments, the dosage of the chemically programmed neutrophils or pharmaceutical formulation thereof can be about 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, or about 1 million neutrophils/kg. In some embodiments, the amount of the pharmaceutical formulation can be 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, or about 1 μg pharmaceutical formulation/kg bodyweight.

Kits Containing the Chemically Programmed Neutrophils and/or Pharmaceutical Formulations Thereof

The chemically programmed neutrophils or pharmaceutical formulations thereof described herein can be presented as a combination kit. As used herein, the terms “combination kit” or “kit of parts” refers to the chemically programmed neutrophils or pharmaceutical formulations thereof and compositions and pharmaceutical formulations thereof described herein and additional components that are used to package, sell, market, deliver, and/or administer the combination of elements or a single element, such as the active ingredient, contained therein. Such additional components include but are not limited to, packaging, syringes, blister packages, bottles, and the like. When one or more of the components (e.g. active agents) contained in the kit are administered simultaneously, the combination kit can contain the active agents in a single pharmaceutical formulation (e.g. a tablet) or in separate pharmaceutical formulations.

The combination kit can contain each agent, compound, pharmaceutical formulation or component thereof described herein, in separate compositions or pharmaceutical formulations. The separate compositions or pharmaceutical formulations can be contained in a single package or in separate packages within the kit. Also provided in some embodiments, are buffers, diluents, solubilization reagents, cell culture media and other reagents. These additional components can be contained in a single package or in separate packages within the kit.

In some embodiments, the combination kit also includes instructions printed on or otherwise contained in a tangible medium of expression. The instructions can provide information regarding the content of the chemically programmed neutrophils or pharmaceutical formulations thereof and/or other auxiliary and/or secondary agent contained therein, safety information regarding the content of the chemically programmed neutrophils or pharmaceutical formulations thereof and/or other auxiliary and/or secondary agent contained therein, information regarding the dosages, indications for use, and/or recommended treatment regimen(s) for the chemically programmed neutrophils or pharmaceutical formulations thereof and/or other auxiliary and/or secondary agent contained therein. In some embodiments, the instructions can provide directions for administering chemically programmed neutrophils or pharmaceutical formulations thereof and/or other auxiliary and/or secondary agent to a subject having or suspected of having non-resolving inflammation and/or related diseases or conditions, including but not limited to atherosclerosis, cardiovascular disease, stroke, myocardial infarction, a neurological disease, and/or a symptom thereof.

### 4-PBA Pharmaceutical Formulations

As noted herein 4-PBA can program neutrophils ex vivo. Also described herein are pharmaceutical formulations that can contain a therapeutically effective amount of 4-PBA and a pharmaceutically acceptable carrier. The 4-PBA pharmaceutical formulations described herein can be administered to a subject in need thereof to treat and/or prevent non-resolving inflammation and/or related diseases or conditions, including but not limited to atherosclerosis, cardiovascular disease, stroke, myocardial infarction, a neurological disease, and/or a symptom thereof in the subject in need thereof.

### Pharmaceutically Acceptable Carriers and Auxiliary Ingredients and Agents

The pharmaceutical formulations containing a therapeutically effective amount of 4-PBA can further include a pharmaceutically acceptable carrier. Suitable pharmaceutically acceptable carriers include, but are not limited to, water, salt solutions, alcohols, gum arabic, vegetable oils, benzyl alcohols, polyethylene glycols, gelatin, carbohydrates such as lactose, amylose or starch, magnesium stearate, talc, silicic acid, viscous paraffin, perfume oil, fatty acid esters, hydroxy methylcellulose, and polyvinyl pyrrolidone, which do not deleteriously react with the active composition.

The 4-PBA pharmaceutical formulations can be sterilized, and if desired, mixed with auxiliary agents, such as lubricants, preservatives, stabilizers, wetting agents, emulsifiers, salts for influencing osmotic pressure, buffers, coloring, flavoring and/or aromatic substances, and the like which do not deleteriously react with the active composition.

In addition to the therapeutically effective amount of 4-PBA, the pharmaceutical formulation can also include an effective amount of an auxiliary active agent, including but not limited to, DNA, RNA, amino acids, peptides, polypeptides, antibodies, aptamers, ribozymes, guide sequences for ribozymes that inhibit translation or transcription of essential tumor proteins and genes, hormones, immunomodulators, antipyretics, anxiolytics, antipsychotics, analgesics, antispasmodics, anti-inflammatories, anti-histamines, anti-infectives, chemotherapeutics and combinations thereof.

### Effective Amounts of 4-PBA and Auxiliary Agents

The 4-PBA pharmaceutical formulations can contain a therapeutically effective amount of 4-PBA. In some embodiments the pharmaceutical formulations can also include a therapeutically effective amount of an auxiliary agent. In some embodiments, the therapeutically effective amount of 4-PBA can range from about 0.1 μg/kg to about 1,000 μg/kg, such as 0.1, 0.2, 0.3, 0.4, 0.5, 0.6, 0.7, 0.8, 0.9, 1, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, to/or 1000 μg/kg. In further embodiments, the therapeutically effective amount of 4-PBA can range from 1 ng/kg bodyweight to about 0.1 mg/kg bodyweight, such as about 1, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, to/or 1000 ng/kg bodyweight, and/or such as 1, 10, 20, 30, 40, 50, 60, 70, 80, 90, to/or 100 μg/kg bodyweight. The therapeutically effective amount of 4-PBA can range from about 1 pg to about 10 g, such as about 1, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, to/or 1000 pg, ng, μg, or mg, or about 1, 1.25, 1.5, 1.75, 2, 2.25, 2.5, 2.75, 3, 3.25, 3.5, 3.75, 4, 4.25, 4.5, 4.75, 5, 5.25, 5.5, 5.75, 6, 6.25, 6.5, 6.75, 7, 7.25, 7.5, 7.75, 8, 8.25, 8.5, 8.75, 9, 9.25, 9.5, 9.75, to/or 10 g. In some embodiments, the therapeutically effective amount of 4-PBA can range from about 10 nL to about 10 mL, such as about 1, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, to/or 1000 nL or μL, or about or about 1, 1.25, 1.5, 1.75, 2, 2.25, 2.5, 2.75, 3, 3.25, 3.5, 3.75, 4, 4.25, 4.5, 4.75, 5, 5.25, 5.5, 5.75, 6, 6.25, 6.5, 6.75, 7, 7.25, 7.5, 7.75, 8, 8.25, 8.5, 8.75, 9, 9.25, 9.5, 9.75, to/or 10 mL. In some embodiments, the therapeutically effective amount of 4-PBA can range from about 10 nL to about 1 μL, such as about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, to/or 1000 nL. For some embodiments, the therapeutically effective amount of 4-PBA can range from about 1 ng to about 1,000 μg per injection, such as about 1, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, to/or 1000 ng or μg per injection, if administered via injection. In some embodiments, the therapeutically effective amount of 4-PBA can be from about 1 to about 1,000 micrograms per injection, such as about 1, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, to/or 1000 μg, such as for a systemically administered injection. In additional embodiments, the therapeutically effective amount of 4-PBA can range from about 100 to about 5,000 μL per injection, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, 1100, 1110, 1120, 1130, 1140, 1150, 1160, 1170, 1180, 1190, 1200, 1210, 1220, 1230, 1240, 1250, 1260, 1270, 1280, 1290, 1300, 1310, 1320, 1330, 1340, 1350, 1360, 1370, 1380, 1390, 1400, 1410, 1420, 1430, 1440, 1450, 1460, 1470, 1480, 1490, 1500, 1510, 1520, 1530, 1540, 1550, 1560, 1570, 1580, 1590, 1600, 1610, 1620, 1630, 1640, 1650, 1660, 1670, 1680, 1690, 1700, 1710, 1720, 1730, 1740, 1750, 1760, 1770, 1780, 1790, 1800, 1810, 1820, 1830, 1840, 1850, 1860, 1870, 1880, 1890, 1900, 1910, 1920, 1930, 1940, 1950, 1960, 1970, 1980, 1990, 2000, 2010, 2020, 2030, 2040, 2050, 2060, 2070, 2080, 2090, 2100, 2110, 2120, 2130, 2140, 2150, 2160, 2170, 2180, 2190, 2200, 2210, 2220, 2230, 2240, 2250, 2260, 2270, 2280, 2290, 2300, 2310, 2320, 2330, 2340, 2350, 2360, 2370, 2380, 2390, 2400, 2410, 2420, 2430, 2440, 2450, 2460, 2470, 2480, 2490, 2500, 2510, 2520, 2530, 2540, 2550, 2560, 2570, 2580, 2590, 2600, 2610, 2620, 2630, 2640, 2650, 2660, 2670, 2680, 2690, 2700, 2710, 2720, 2730, 2740, 2750, 2760, 2770, 2780, 2790, 2800, 2810, 2820, 2830, 2840, 2850, 2860, 2870, 2880, 2890, 2900, 2910, 2920, 2930, 2940, 2950, 2960, 2970, 2980, 2990, 3000, 3010, 3020, 3030, 3040, 3050, 3060, 3070, 3080, 3090, 3100, 3110, 3120, 3130, 3140, 3150, 3160, 3170, 3180, 3190, 3200, 3210, 3220, 3230, 3240, 3250, 3260, 3270, 3280, 3290, 3300, 3310, 3320, 3330, 3340, 3350, 3360, 3370, 3380, 3390, 3400, 3410, 3420, 3430, 3440, 3450, 3460, 3470, 3480, 3490, 3500, 3510, 3520, 3530, 3540, 3550, 3560, 3570, 3580, 3590, 3600, 3610, 3620, 3630, 3640, 3650, 3660, 3670, 3680, 3690, 3700, 3710, 3720, 3730, 3740, 3750, 3760, 3770, 3780, 3790, 3800, 3810, 3820, 3830, 3840, 3850, 3860, 3870, 3880, 3890, 3900, 3910, 3920, 3930, 3940, 3950, 3960, 3970, 3980, 3990, 4000, 4010, 4020, 4030, 4040, 4050, 4060, 4070, 4080, 4090, 4100, 4110, 4120, 4130, 4140, 4150, 4160, 4170, 4180, 4190, 4200, 4210, 4220, 4230, 4240, 4250, 4260, 4270, 4280, 4290, 4300, 4310, 4320, 4330, 4340, 4350, 4360, 4370, 4380, 4390, 4400, 4410, 4420, 4430, 4440, 4450, 4460, 4470, 4480, 4490, 4500, 4510, 4520, 4530, 4540, 4550, 4560, 4570, 4580, 4590, 4600, 4610, 4620, 4630, 4640, 4650, 4660, 4670, 4680, 4690, 4700, 4710, 4720, 4730, 4740, 4750, 4760, 4770, 4780, 4790, 4800, 4810, 4820, 4830, 4840, 4850, 4860, 4870, 4880, 4890, 4900, 4910, 4920, 4930, 4940, 4950, 4960, 4970, 4980, 4990, to/or 5000 μL per injection, such as for a systemically administered injection.

In embodiments where there is an auxiliary active agent contained in the pharmaceutical formulation in addition to the 4-PBA, the therapeutically effective amount of the auxiliary active agent will vary depending on the auxiliary active agent. In some embodiments, the effective amount of the auxiliary active agent ranges from 0.001 micrograms to about 1 milligram, such as 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, or 1 micrograms or milligrams. In other embodiments, the effective amount of the auxiliary active agent ranges from about 0.01 IU to about 1000 IU, such as about 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or about 1000 IU. In further embodiments, the effective amount of the auxiliary active agent ranges from 0.001 mL to about 1 mL, such as about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, or about 1 mL. In yet other embodiments, the effective amount of the auxiliary active agent ranges from about 1% to about 50% w/w, v/v, or w/v, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50% w/w, v/v, or w/v of the total pharmaceutical formulation.

### Dosage Forms

In some embodiments, the 4-PBA pharmaceutical formulations described herein may be in a dosage form. The dosage forms can be adapted for administration by any appropriate route. Appropriate routes include, but are not limited to, oral (including buccal or sublingual), rectal, epidural, intracranial, intraocular, inhaled, intranasal, topical (including buccal, sublingual, or transdermal), vaginal, intraurethral, parenteral, intracranial, subcutaneous, intramuscular, intravenous, intraperitoneal, intradermal, intraosseous, intracardiac, intraarticular, intracavernous, intrathecal, intravitreal, intracerebral, gingival, subgingival, intracerebroventricular, and intradermal. Such formulations may be prepared by any method known in the art.

Dosage forms adapted for oral administration can be discrete dosage units such as capsules, pellets or tablets, powders or granules, solutions, or suspensions in aqueous or non-aqueous liquids; edible foams or whips, or in oil-in-water liquid emulsions or water-in-oil liquid emulsions. In some embodiments, the 4-PBA pharmaceutical formulations adapted for oral administration also include one or more agents which flavor, preserve, color, or help disperse the pharmaceutical formulation. Dosage forms prepared for oral administration can also be in the form of a liquid solution that can be delivered as foam, spray, or liquid solution. In some embodiments, the oral dosage form can contain about 1 ng to 1000 g of a pharmaceutical formulation containing a therapeutically effective amount or an appropriate fraction thereof of 4-PBA. The oral dosage form can be administered to a subject in need thereof.

Where appropriate, the dosage forms described herein can be microencapsulated. The dosage form can also be prepared to prolong or sustain the release of any ingredient. In some embodiments, 4-PBA can be the ingredient whose release is delayed. In other embodiments, the release of an optionally included auxiliary ingredient is delayed. Suitable methods for delaying the release of an ingredient include, but are not limited to, coating or embedding the ingredients in material in polymers, wax, gels, and the like. Delayed release dosage formulations can be prepared as described in standard references such as “Pharmaceutical dosage form tablets,” eds. Liberman et. al. (New York, Marcel Dekker, Inc., 1989), “Remington—The science and practice of pharmacy”, 20th ed., Lippincott Williams & Wilkins, Baltimore, Md., 2000, and “Pharmaceutical dosage forms and drug delivery systems”, 6th Edition, Ansel et al., (Media, PA: Williams and Wilkins, 1995). These references provide information on excipients, materials, equipment, and processes for preparing tablets and capsules and delayed release dosage forms of tablets and pellets, capsules, and granules. The delayed release can be anywhere from about an hour to about 3 months or more.

Examples of suitable coating materials include, but are not limited to, cellulose polymers such as cellulose acetate phthalate, hydroxypropyl cellulose, hydroxypropyl methylcellulose, hydroxypropyl methylcellulose phthalate, and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Coatings may be formed with a different ratio of water-soluble polymer, water insoluble polymers, and/or pH dependent polymers, with or without water insoluble/water soluble non-polymeric excipient, to produce the desired release profile. The coating is either performed on the dosage form (matrix or simple) which includes, but is not limited to, tablets (compressed with or without coated beads), capsules (with or without coated beads), beads, particle compositions, “ingredient as is” formulated as, but not limited to, suspension form or as a sprinkle dosage form.

Dosage forms adapted for topical administration can be formulated as ointments, creams, suspensions, lotions, powders, solutions, pastes, gels, sprays, aerosols, or oils. In some embodiments for treatments of the eye or other external tissues, for example the mouth or the skin, the pharmaceutical formulations are applied as a topical ointment or cream. When formulated in an ointment, 4-PBA, auxiliary active ingredient, and/or pharmaceutically acceptable salt thereof can be formulated with a paraffinic or water-miscible ointment base. In some embodiments, the active ingredient can be formulated in a cream with an oil-in-water cream base or a water-in-oil base. Dosage forms adapted for topical administration in the mouth include lozenges, pastilles, and mouth washes.

Dosage forms adapted for nasal or inhalation administration include aerosols, solutions, suspension drops, gels, or dry powders. In some embodiments, 4-PBA, auxiliary active ingredient, and/or pharmaceutically acceptable salt thereof in a dosage form adapted for inhalation is in a particle-size-reduced form that is obtained or obtainable by micronization. In some embodiments, the particle size of the size reduced (e.g. micronized) compound or salt or solvate thereof, is defined by a D50 value of about 0.5 to about 10 microns as measured by an appropriate method known in the art. Dosage forms adapted for administration by inhalation also include particle dusts or mists. Suitable dosage forms wherein the carrier or excipient is a liquid for administration as a nasal spray or drops include aqueous or oil solutions/suspensions of an active ingredient (e.g. 4-PBA and/or auxiliary active agent), which may be generated by various types of metered dose pressurized aerosols, nebulizers, or insufflators.

In some embodiments, the dosage forms can be aerosol formulations suitable for administration by inhalation. In some of these embodiments, the aerosol formulation can contain a solution or fine suspension of 4-PBA, auxiliary agent thereof, and/or pharmaceutically acceptable salt thereof, and a pharmaceutically acceptable aqueous or non-aqueous solvent. Aerosol formulations can be presented in single or multi-dose quantities in sterile form in a sealed container. For some of these embodiments, the sealed container is a single dose or multi-dose nasal or an aerosol dispenser fitted with a metering valve (e.g. metered dose inhaler), which is intended for disposal once the contents of the container have been exhausted.

Where the aerosol dosage form is contained in an aerosol dispenser, the dispenser contains a suitable propellant under pressure, such as compressed air, carbon dioxide, or an organic propellant, including but not limited to a hydrofluorocarbon. The aerosol formulation dosage forms in other embodiments are contained in a pump-atomizer. The pressurized aerosol formulation can also contain a solution or a suspension of 4-PBA. In further embodiments, the aerosol formulation can also contain co-solvents and/or modifiers incorporated to improve, for example, the stability and/or taste and/or fine particle mass characteristics (amount and/or profile) of the formulation. Administration of the aerosol formulation can be once daily or several times daily, for example 2, 3, 4, or 8 times daily, in which 1, 2, or 3 doses are delivered each time.

For some dosage forms suitable and/or adapted for inhaled administration, the pharmaceutical formulation is a dry powder inhalable formulation. In addition to the 4-PBA, an auxiliary active ingredient, and/or pharmaceutically acceptable salt thereof, such a dosage form can contain a powder base such as lactose, glucose, trehalose, manitol, and/or starch. In some of these embodiments, 4-PBA, auxiliary active ingredient, and/or pharmaceutically acceptable salt thereof is in a particle-size reduced form. In further embodiments, a performance modifier, such as L-leucine or another amino acid, cellobiose octaacetate, and/or metals salts of stearic acid, such as magnesium or calcium stearate.

In some embodiments, the aerosol dosage forms can be arranged so that each metered dose of aerosol contains a predetermined amount of an active ingredient, such as 4-PBA.

Dosage forms adapted for vaginal administration can be presented as pessaries, tampons, creams, gels, pastes, foams, or spray formulations. Dosage forms adapted for rectal administration include suppositories or enemas.

Dosage forms adapted for parenteral administration and/or adapted for any type of injection (e.g. intravenous, intraperitoneal, subcutaneous, intramuscular, intradermal, intraosseous, epidural, intracardiac, intraarticular, intracavernous, gingival, subgingival, intrathecal, intravireal, intracerebral, and intracerebroventricular) can include aqueous and/or non-aqueous sterile injection solutions, which can contain anti-oxidants, buffers, bacteriostats, solutes that render the composition isotonic with the blood of the subject, and aqueous and non-aqueous sterile suspensions, which can include suspending agents and thickening agents. The dosage forms adapted for parenteral administration can be presented in a single-unit dose or multi-unit dose containers, including but not limited to sealed ampoules or vials. The doses can be lyophilized and resuspended in a sterile carrier to reconstitute the dose prior to administration. Extemporaneous injection solutions and suspensions can be prepared in some embodiments, from sterile powders, granules, and tablets.

Dosage forms adapted for ocular administration can include aqueous and/or non-aqueous sterile solutions that can optionally be adapted for injection, and which can optionally contain anti-oxidants, buffers, bacteriostats, solutes that render the composition isotonic with the eye or fluid contained therein or around the eye of the subject, and aqueous and non-aqueous sterile suspensions, which can include suspending agents and thickening agents.

For some embodiments, the dosage form contains a predetermined amount of 4-PBA per unit dose. In some embodiments, the predetermined amount of 4-PBA is a therapeutically effective amount of 4-PBA is effective to treat and/or prevent non-resolving inflammation and/or related diseases or conditions, including but not limited to atherosclerosis, cardiovascular disease, stroke, myocardial infarction, a neurological disease, and/or a symptom thereof in a subject in need thereof. In other embodiments, the predetermined amount of 4-PBA can be an appropriate fraction of the therapeutically effective amount of the active ingredient (e.g. 4-PBA and/or auxiliary active agent). Such unit doses may therefore be administered once or more than once a day. Such pharmaceutical formulations may be prepared by any of the methods well known in the art.

### Methods of Using the 4-PBA Pharmaceutical Formulations

The 4-PBA pharmaceutical formulations described herein can be used for the treatment and/or prevention of a disease, disorder, syndrome, or a symptom thereof in a subject. In some embodiments, the 4-PBA pharmaceutical formulation described herein can be used to treat and/or prevent non-resolving inflammation and/or related diseases or conditions, including but not limited to atherosclerosis, cardiovascular disease, stroke, myocardial infarction, neurological disease, and/or a symptom thereof in the subject in need thereof.

An amount of a 4-PBA pharmaceutical formulation described herein can be administered to a subject in need thereof one or more times per day, week, month, or year. In some embodiments, the amount administered can be the therapeutically effective amount of the 4-PBA pharmaceutical formulation described herein. For example, the 4-PBA pharmaceutical formulation described herein can be administered in a daily dose. This amount may be given in a single dose per day. In other embodiments, the daily dose may be administered over multiple doses per day, in which each contains a fraction of the total daily dose to be administered (sub-doses). In some embodiments, the number of doses delivered per day is 2, 3, 4, 5, or 6. In further embodiments, the 4-PBA pharmaceutical formulation described herein can be administered one or more times per week, such as 1, 2, 3, 4, 5, or 6 times per week. In other embodiments, the 4-PBA pharmaceutical formulation described herein can be administered one or more times per month, such as 1 to 5 times per month. In still further embodiments, the 4-PBA pharmaceutical formulation described herein can be administered one or more times per year, such as 1 to 11 times per year.

The 4-PBA pharmaceutical formulation described herein can be co-administered with a secondary agent by any convenient route. The secondary agent is a separate compound and/or pharmaceutical formulation from the 4-PBA pharmaceutical formulation described herein. The secondary agent can be administered simultaneously with 4-PBA pharmaceutical formulation described herein. The secondary agent can be administered sequentially with the 4-PBA pharmaceutical formulation described herein. Suitable secondary agents include, but are not limited to, DNA, RNA, amino acids, peptides, polypeptides, antibodies, aptamers, ribozymes, guide sequences for ribozymes that inhibit translation or transcription of essential tumor proteins and genes, hormones, immunomodulators, antipyretics, anxiolytics, antipsychotics, analgesics, antispasmodics, anti-inflammatories, anti-histamines, anti-infectives, and chemotherapeutics.

In embodiments where the 4-PBA pharmaceutical formulation described herein are simultaneously co-administered with a secondary agent, the 4-PBA pharmaceutical formulation described herein can be administered to the subject at substantially the same time as the secondary agent. As used in this context “substantially the same time” refers to administration of the 4-PBA pharmaceutical formulation described herein and a secondary agent where the period of time between administration of the 4-PBA pharmaceutical formulation described herein and the secondary agent is between 0 and 10 minutes.

In embodiments where the 4-PBA pharmaceutical formulation described herein is/are sequentially co-administered with a secondary agent, the 4-PBA pharmaceutical formulation described herein can be administered first, and followed by administration of the secondary agent after a period of time. In other embodiments where the 4-PBA pharmaceutical formulation described herein is/are sequentially co-administered with a secondary agent, the secondary agent can be administered first, and followed by administration of the 4-PBA pharmaceutical formulation described herein after a period of time. The period of time between administration of the 4-PBA pharmaceutical formulation described herein and the secondary agent can range from 10 minutes to about 96 hours or more. In some embodiments, the period of time can be about 10 minutes, about 30 minutes, about 1 hour, about 2 hours, about 4 hours, about 6 hours, about 8 hours, about 10 hours, or about 12 hours or more. The sequential administration can be repeated as necessary over the course of the period of treatment.

The amount of the 4-PBA pharmaceutical formulation described herein that can be administered are described elsewhere herein. The amount of the secondary agent will vary depending on the secondary agent. The amount of the secondary agent can be a therapeutically effective amount. In some embodiments, the effective amount of the secondary agent ranges from 0.001 micrograms to about 1 milligram, such as 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, or 1 micrograms or milligrams. In other embodiments, the amount of the secondary agent ranges from about 0.01 IU to about 1000 IU, such as about 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or about 1000 IU. In further embodiments, the amount of the secondary agent ranges from 0.001 mL to about 1 mL, such as about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, or about 1 mL. In yet other embodiments, the amount of the secondary agent ranges from about 1% to about 50% w/w, v/v, or w/v, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50% w/w, v/v, or w/v of the total pharmaceutical formulation.

In some embodiments, the 4-PBA pharmaceutical formulation described herein can be administered to a patient via an injection. Suitable methods of injection include, but are not limited to, intravenous, intraperitoneal, subcutaneous, intramuscular, intradermal, intraosseous, epidural, intracardiac, intraarticular, intracavernous, intrathecal, intravitreal, intracerebral, gingival, subgingival, intranodal, and intracerebroventricular injection. Other suitable methods of administration of the 4-PBA pharmaceutical formulation described herein, but are not limited to, subcutaneous, intravenous, parenteral, and/or oral delivery. In some embodiments, the dosage of 4-PBA pharmaceutical formulation described herein can range from about 0.01 μg/kg bodyweight to about 1 mg/kg bodyweight.

### Kits Containing the 4-PBA Pharmaceutical Formulations

The 4-PBA pharmaceutical formulation described herein described herein can be presented as a combination kit. As used herein, the terms “combination kit” or “kit of parts” refers to the 4-PBA pharmaceutical formulation described herein and additional components that are used to package, sell, market, deliver, and/or administer the combination of elements or a single element, such as the active ingredient, contained therein. Such additional components include but are not limited to, packaging, syringes, blister packages, bottles, and the like. When one or more of the components (e.g. active agents) contained in the kit are administered simultaneously, the combination kit can contain the active agents in a single pharmaceutical formulation (e.g. a tablet) or in separate pharmaceutical formulations.

The 4-PBA combination kit can contain each agent, compound, 4-PBA pharmaceutical formulation or component thereof described herein, in separate compositions or pharmaceutical formulations. The separate compositions or pharmaceutical formulations can be contained in a single package or in separate packages within the kit. Also provided in some embodiments, are buffers, diluents, solubilization reagents, cell culture media and other reagents. These additional components can be contained in a single package or in separate packages within the kit.

In some embodiments, the combination kit also includes instructions printed on or otherwise contained in a tangible medium of expression. The instructions can provide information regarding the content of the 4-PBA pharmaceutical formulation described herein and/or other auxiliary and/or secondary agent contained therein, safety information regarding the 4-PBA pharmaceutical formulation described herein and/or other auxiliary and/or secondary agent contained therein, information regarding the dosages, indications for use, and/or recommended treatment regimen(s) for the 4-PBA pharmaceutical formulation described herein and/or other auxiliary and/or secondary agent contained therein. In some embodiments, the instructions can provide directions for administering the 4-PBA pharmaceutical formulation described herein and/or other auxiliary and/or secondary agent to a subject having or suspected of having non-resolving inflammation and/or related diseases or conditions, including but not limited to atherosclerosis, cardiovascular disease, stroke, myocardial infarction, neurological disease, and/or a symptom thereof.

Without further elaboration, it is believed that one skilled in the art can, based on the description herein, utilize the present disclosure to its fullest extent. It is emphasized that the embodiments of the present disclosure, particularly any “preferred” embodiments, are merely possible examples of the implementations, merely set forth for a clear understanding of the principles of the disclosure. Many variations and modifications may be made to the disclosed embodiment(s) of the disclosure without departing substantially from the spirit and principles of the disclosure. All such modifications and variations are within the scope of this disclosure.

### EXAMPLES

Now having described the embodiments of the present disclosure, in general, the following Examples describe some additional embodiments of the present disclosure. While embodiments of the present disclosure are described in connection with the following examples and the corresponding text and figures, there is no intent to limit embodiments of the present disclosure to this description. On the contrary, the intent is to cover all alternatives, modifications, and equivalents included within the spirit and scope of embodiments of the present disclosure. The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

### Example 1

Atherosclerosis and related cardiovascular complications are leading causes of morbidity and mortality world-wide with serious economic and health tolls. One of the key risk factors for atherosclerosis is the establishment of non-resolving inflammation. However, the limited understanding of underlying mechanisms presents a major road-block for effective prevention and treatment (1-3).

Emerging studies suggest that the lack of inflammation resolution during atherosclerosis may occur due to re-programing and polarization of innate leukocytes under persistent low-grade inflammatory challenges (4, 5). Some of the well-studied aspects of polarized “memory” macrophages during atherosclerosis favor foam cell formation, reduce efferocytosis, and elevate necrosis, which collectively contribute to the pathogenesis of atherosclerosis (6-8). In addition to the well-studied macrophages during atherosclerosis, neutrophils may also be involved through less-characterized mechanisms (9-11). Neutrophils constitute 50-70% of circulating white blood cells and have been shown to be elevated in circulating blood as well as atherosclerotic plaques from human patients and experimental animals with unstable plaques (12-17). Although the correlation of higher neutrophil populations with increased risks of unstable atherosclerotic plaques has been increasingly appreciated, it is poorly understood, however, regarding how neutrophils are differentially polarized and contribute to atherosclerosis under chronic inflammatory conditions.

Using a previously developed murine model of low-grade inflammation and atherosclerosis that was developed through repetitive injections of subclinical dose lipopolysaccharide (LPS), the role that differentially re-programmed neutrophils under chronic inflammatory condition can play during the pathogenesis of atherosclerosis was examined. Briefly, the unique polarization of neutrophils by subclinical low dose LPS both in vitro and in vivo was examined, as well as the underlying molecular and cellular mechanisms.

Through transfusing uniquely programmed neutrophils into recipient mice, the role of re-programmed neutrophils during the pathogenesis of atherosclerosis and plaque stability was directly examined. The disruption of peroxisome homeostasis by subclinical low-dose LPS is uniquely responsible for the inflammatory polarization of neutrophils was identified. The efficacy of restoring neutrophil peroxisome homeostasis with 4-PBA, and the therapeutic potential of transfusing 4-PBA programmed neutrophils in treating experimental atherosclerosis was examined.

### Results

Reduced Plaque Stability in Mice Challenged with Subclinical Dose of Endotoxin.

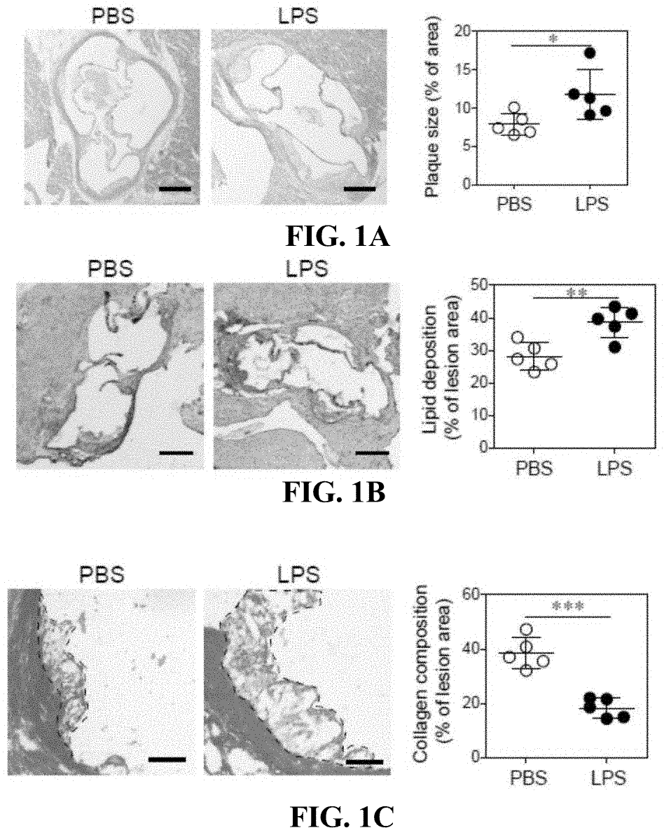

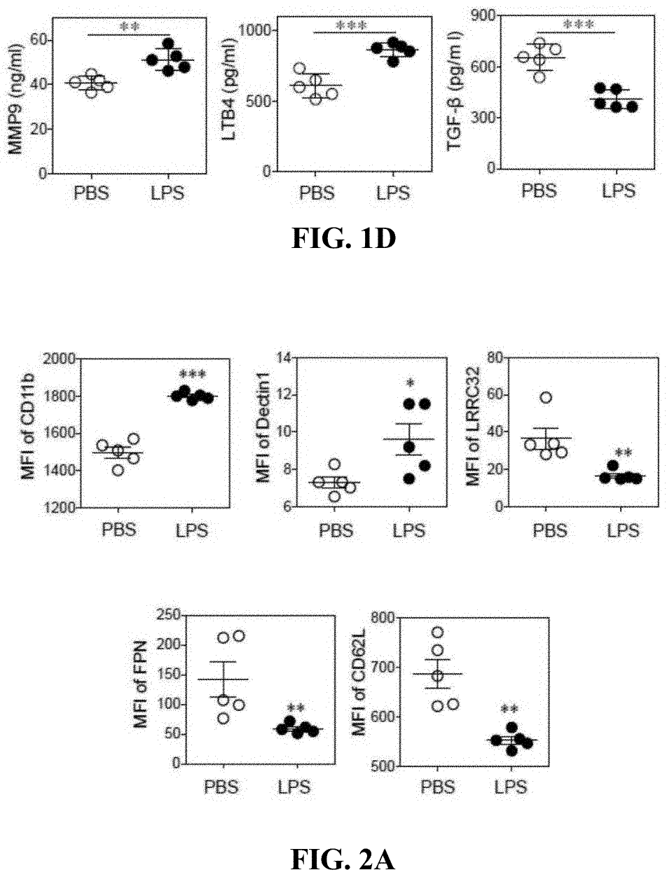

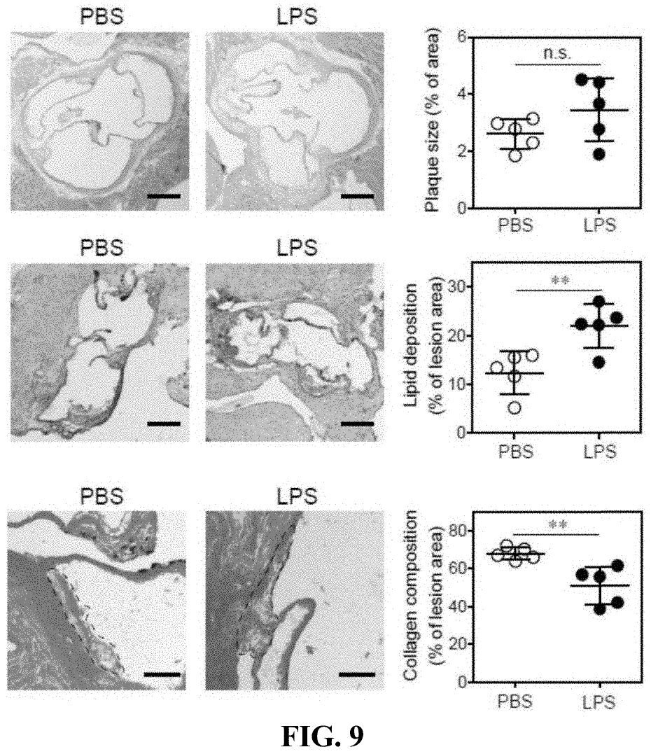

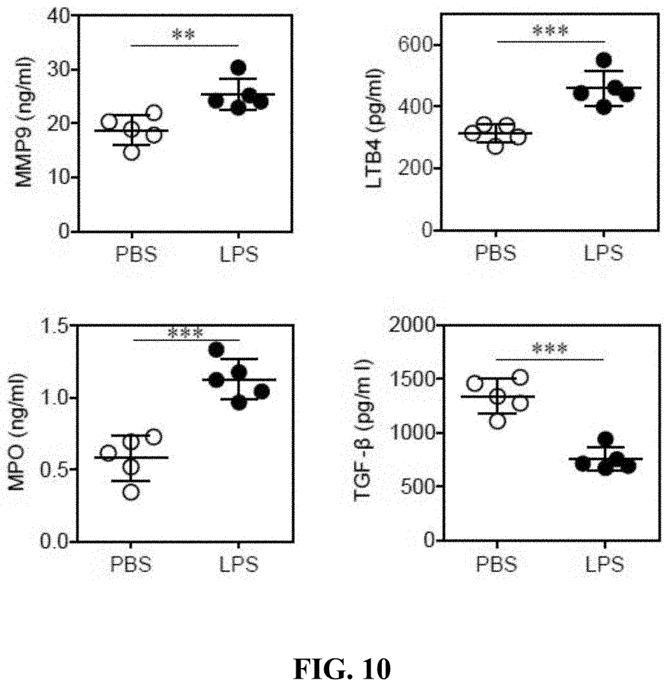

It was first confirmed that that subclinical low dose LPS exacerbates atherosclerosis progression through Oil-Red 0 and H & E staining (5). High fat diet (HFD)-fed ApoE−/− mice injected weekly with subclinical low dose LPS (5 ng/kg body weight) for 4 weeks developed significantly larger atherosclerotic plaques as compared with mice injected with PBS (FIGS. 1A and 1B). Most noteworthy, chronic injection of subclinical low dose LPS significantly reduced the plaque stability as reflected in significantly reduced collagen content within the plaque (FIG. 1C). Matrix metallopeptidases such as MMP9 responsible for degrading collagens as well as inflammatory lipid mediators such as LTB4 have been closely associated with reduced plaque stability (18-21). The levels of MMP9 were then tested and a significant elevation of plasma MMP9 and LTB4 levels in mice injected with LPS was observed as compared to mice injected with PBS (FIG. 1D). LPS administration also induced remarkable elevation of circulating MPO (FIG. 8), another pro-inflammatory mediator promoting plaque instability (22). On the other hand, the circulating levels of anti-inflammatory mediator TGFβ were significantly reduced in mice injected with low dose LPS (FIG. 1D). Similar effects were observed in regular chow diet (RD)-fed mice injected with low dose LPS (FIGS. 9-10).

### Inflammatory Polarization of Neutrophils During Atherosclerosis.

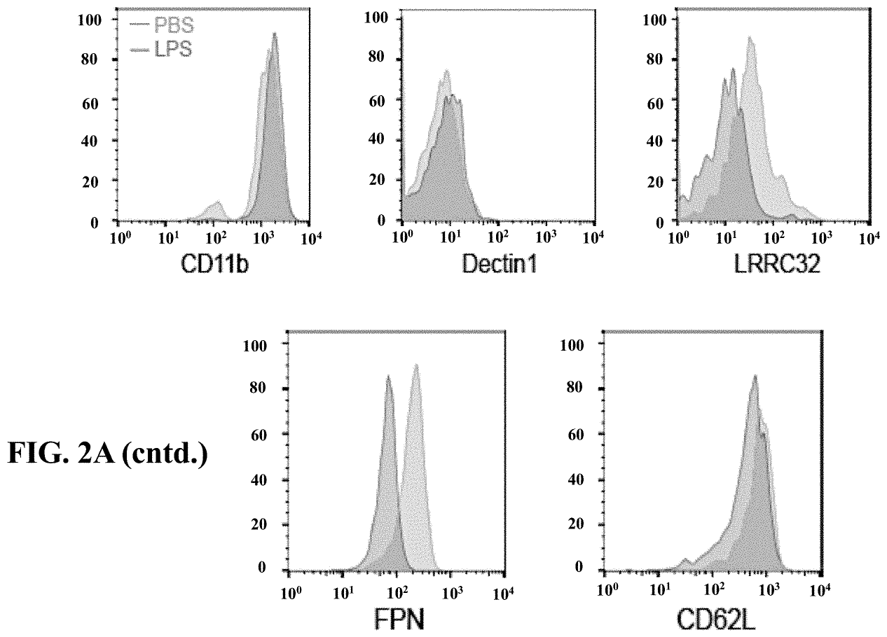

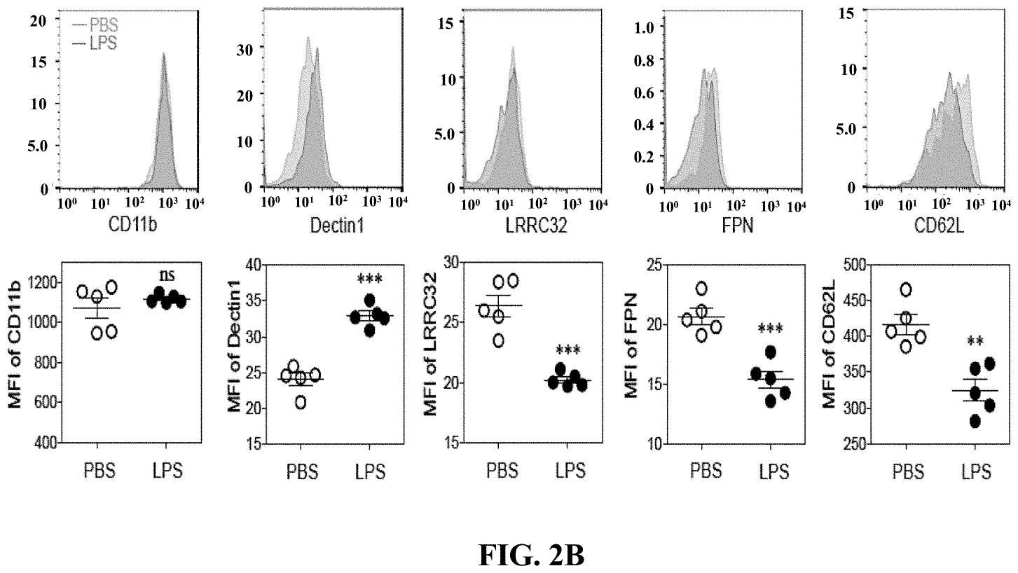

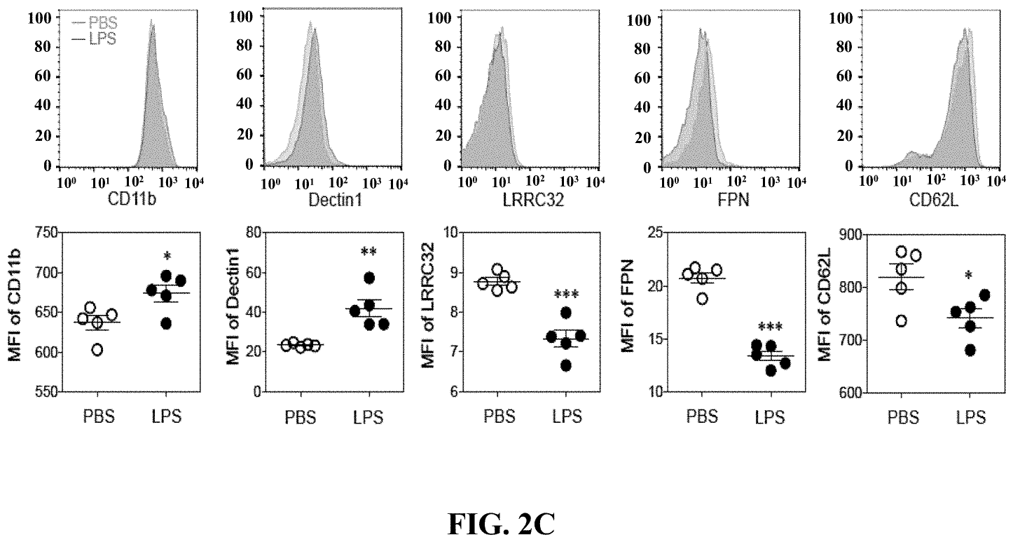

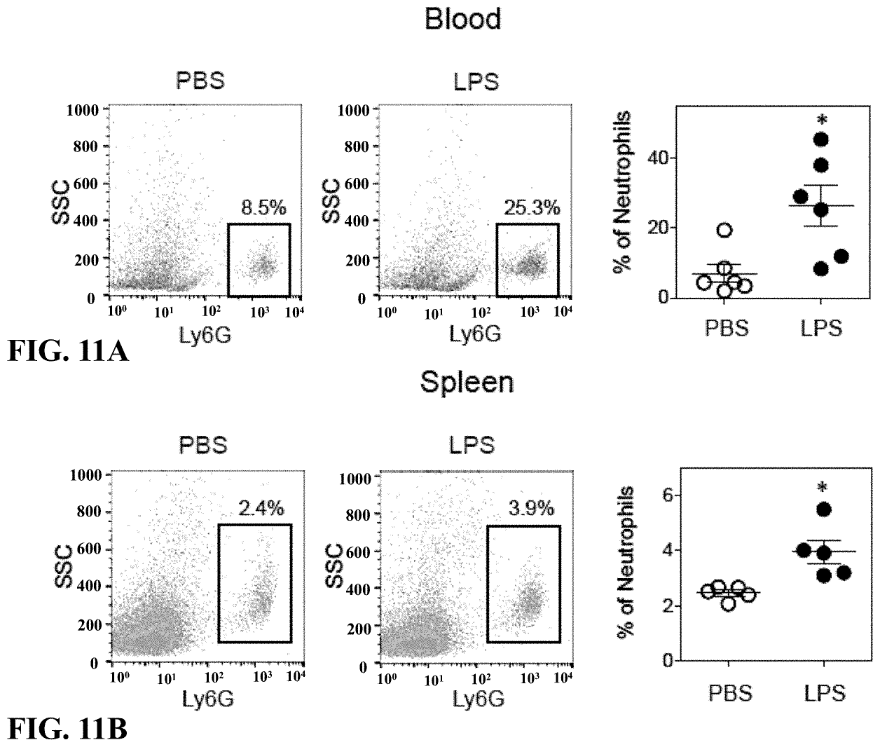

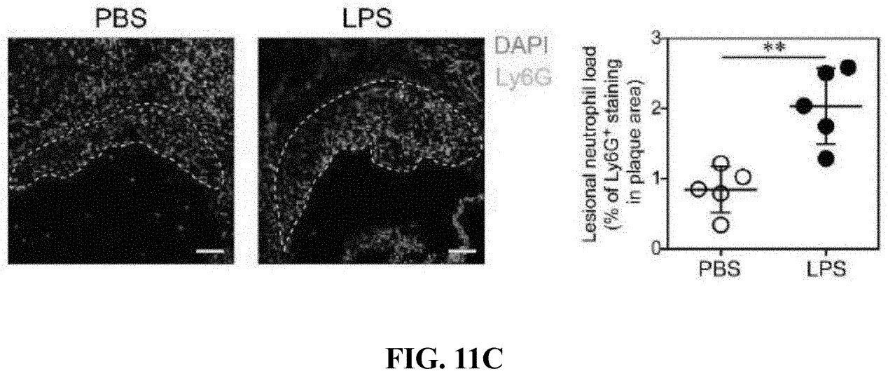

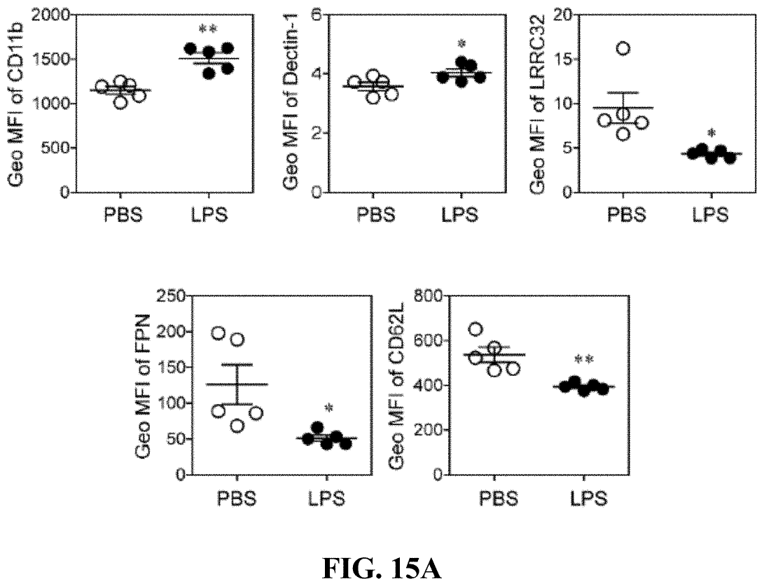

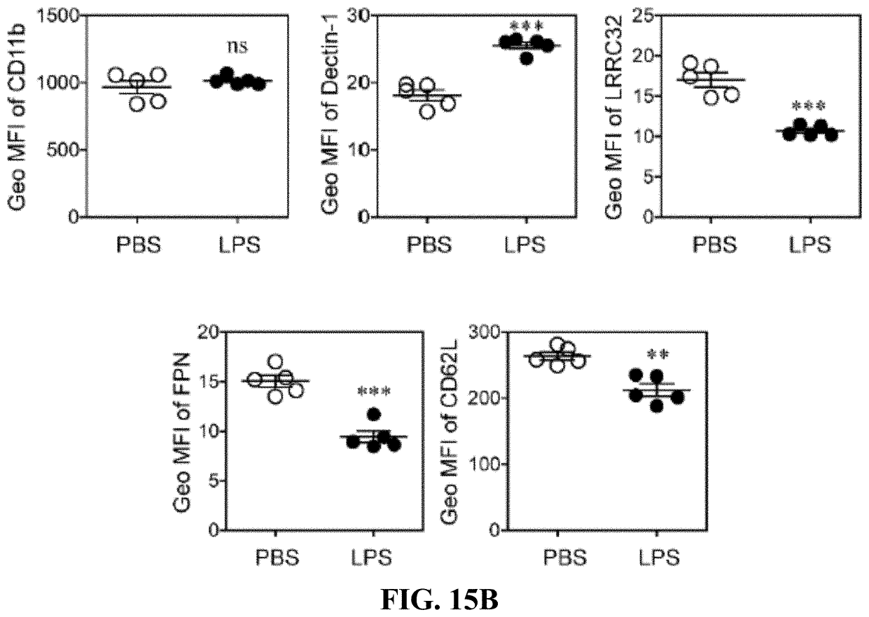

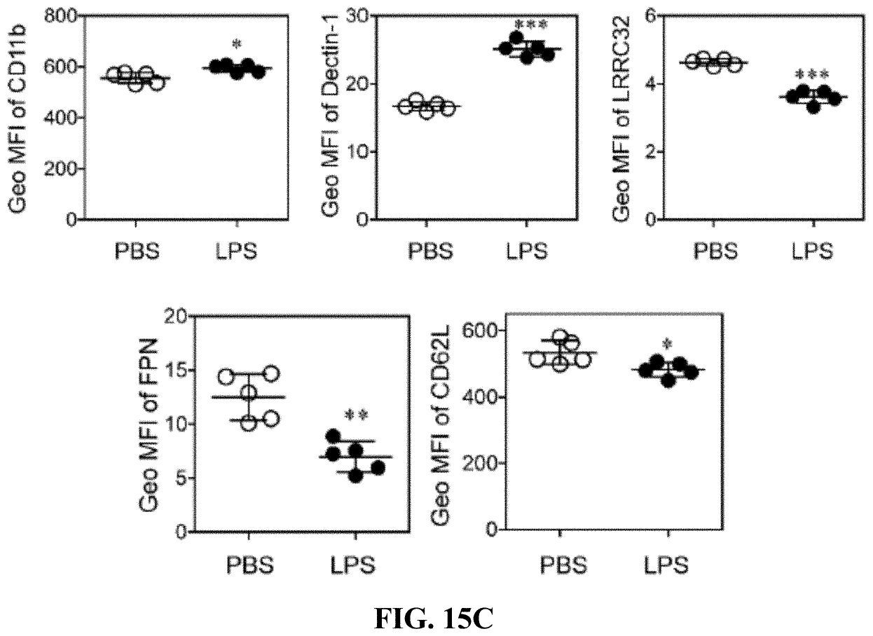

Among innate leukocytes, neutrophils are the primary producer of MMP9 and LTB4 (23, 24). Although elevated MMP9 and LTB4 have been well-associated with unstable atherosclerotic plaques, the involvement of neutrophils during the secretion of MMP9 and LTB4 related with atherosclerosis has not been studied. The neutrophil activation status within mice injected with subclinical low dose LPS was tested trough examining key surface activation markers by flow cytometry. Select inflammatory markers such as CD11b and Dectin1 were significantly elevated in LPS-injected mice as compared to PBS-injected mice (FIGS. 2A-2C). In addition, these neutrophils had a significant reduction of surface-attached CD62L (FIGS. 2A-2C), which is another classical indicator of neutrophil activation (25). A similar trend in neutrophils harvested from circulating blood (FIG. 2A), spleen (FIG. 2B), and bone marrow (FIG. 2C). In addition to increased activation, higher percentages of neutrophils in the blood, spleens, and atherosclerotic plaques of mice chronically injected with LPS was observed (FIGS. 11A-11C). Along with markers of neutrophil activation, the expression of key homeostatic molecules such as leucine-rich repeat containing 32 (LRRC32) and ferroportin (FPN) were examined. LRRC32 is a cell surface conjugated molecule responsible for cleaving latent TGFβ and generating the soluble form of TGFβ (26). FPN is involved in the polarization of innate leukocytes into an anti-inflammatory state through modulating intra-cellular iron content (27). It was observed that neutrophils from mice chronically injected with LPS had significantly reduced surface levels of LLRC32 and FPN (FIGS. 2A-2C). Instead of mean fluorescence intensity (MFI), geometric MFI was used as a parameter to analyze the expressions of tested molecules, demonstrating a similar modulation of neutrophil phenotype after LPS treatment (FIGS. 15A-15C). Together, these data can demonstrate at least an in vivo polarization of neutrophils in mice chronically injected with low dose LPS.

### Mechanisms Underlying the Inflammatory Polarization of Neutrophils.

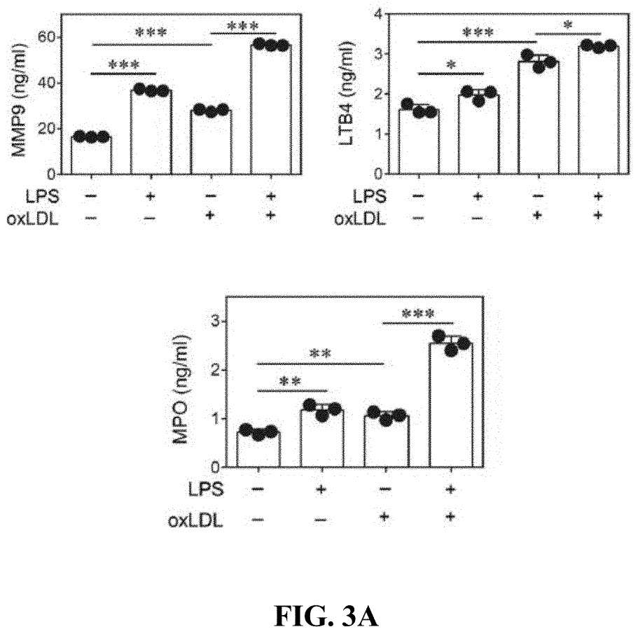

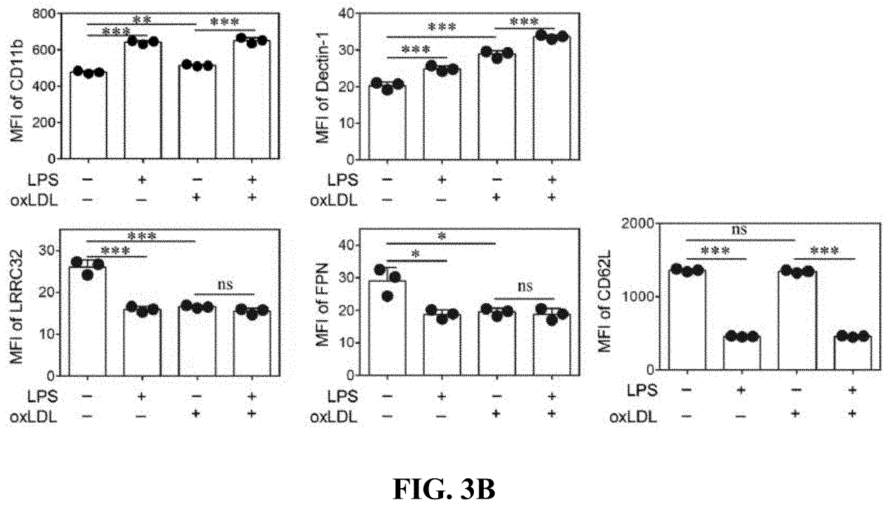

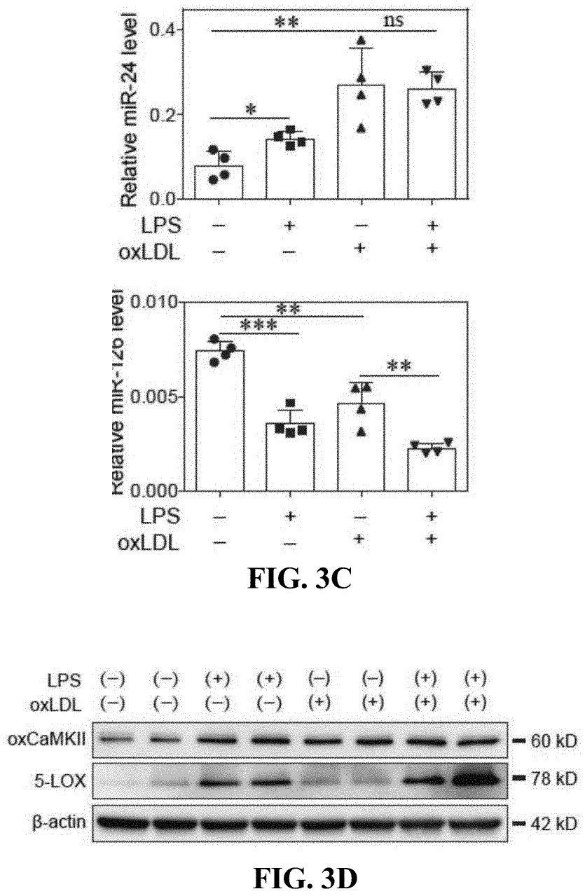

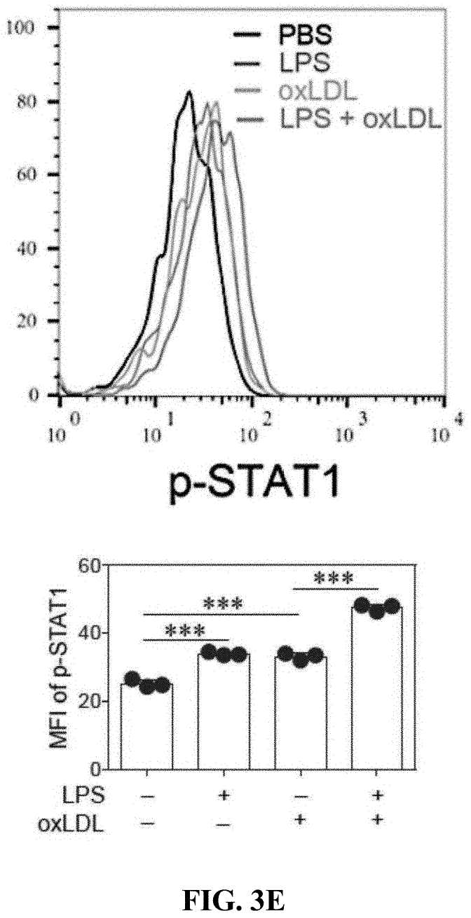

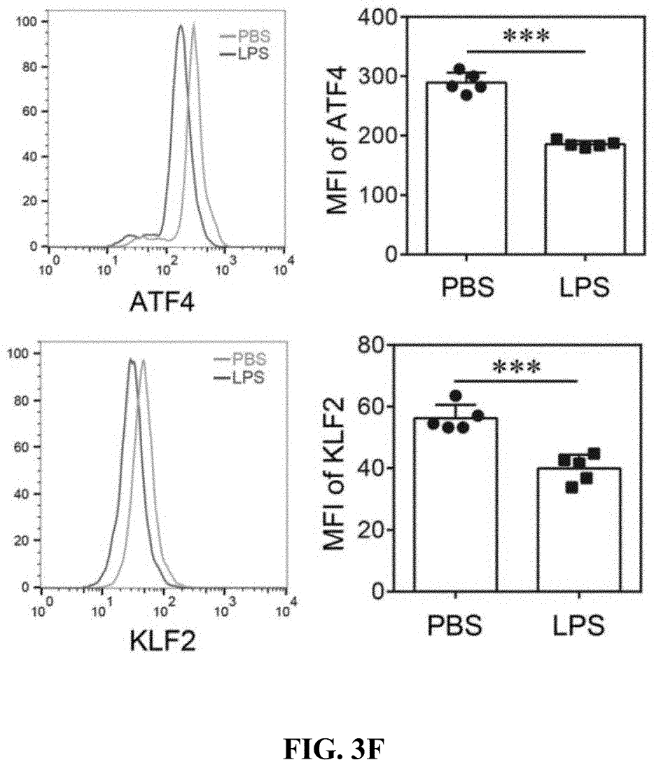

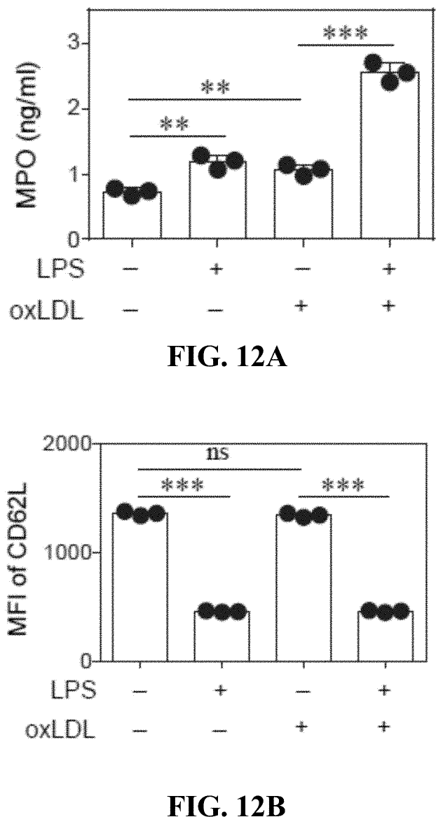

After characterizing the polarization of neutrophils in vivo by subclinical-dose LPS challenge, it was further examined whether low dose LPS may directly polarize neutrophils in vitro. Bone-marrow (BM)-derived neutrophils were cultured with granulocyte colony-stimulating factor (G-CSF) together with or without LPS overnight. The activation status of neutrophils was monitored through assessing inflammatory mediators by enzyme-linked immunosorbent assay (ELISA) as well as key cell surface markers via flow cytometry. Significantly higher levels of MMP9, LTB4 and MPO in the supernatant of neutrophils cultured with low-dose LPS as compared to control neutrophils (those cultured in G-CSF alone) was observed (FIGS. 3A and 12A). Significantly elevated levels of CD11b and Dectin-1, as well as significantly reduced levels of CD62L, LRRC32, and FPN was observed on neutrophils cultured with low-dose LPS (FIGS. 3B and 12B). Coculture of neutrophils with low-dose LPS and oxidized low density lipoprotein (oxLDL) further synergized the induction of MMP9, LTB4 and MPO (FIGS. 3A and 12A). In addition to inflammatory lipid mediators, the expression of selected miRNAs expressed by neutrophils was also examined. It has previously been demonstrated that a subclinical dose LPS can potently induce the expression of miR-24 in monocytes, a critical miRNA involved in propagating the non-resolving inflammation (5). It was observed that neutrophils challenged with a subclinical dose of LPS also express miR-24 (FIG. 3C). Subclinical dose LPS not only induced the inflammatory miR-24, but also potently suppressed the expression of mi-126 (FIG. 3C), a key miRNA involved in tissue homeostasis and vascular integrity (28, 29).

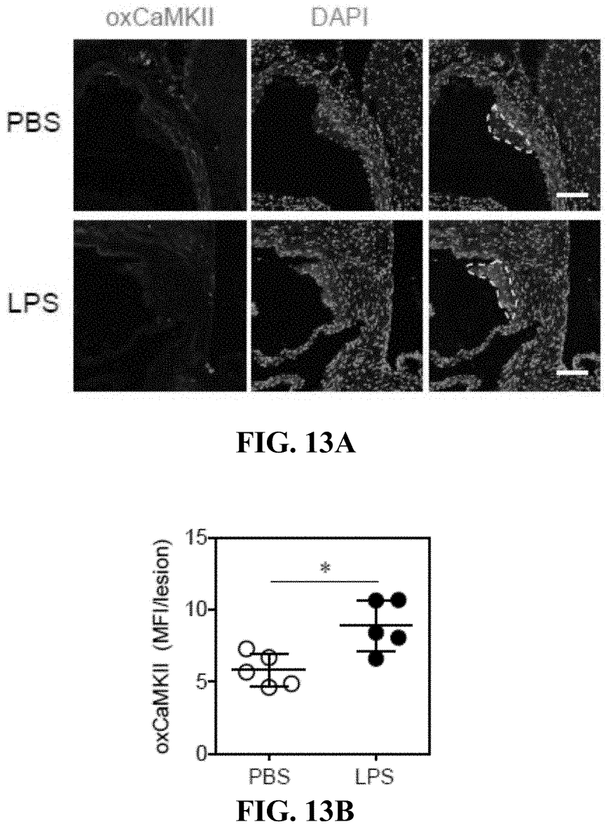

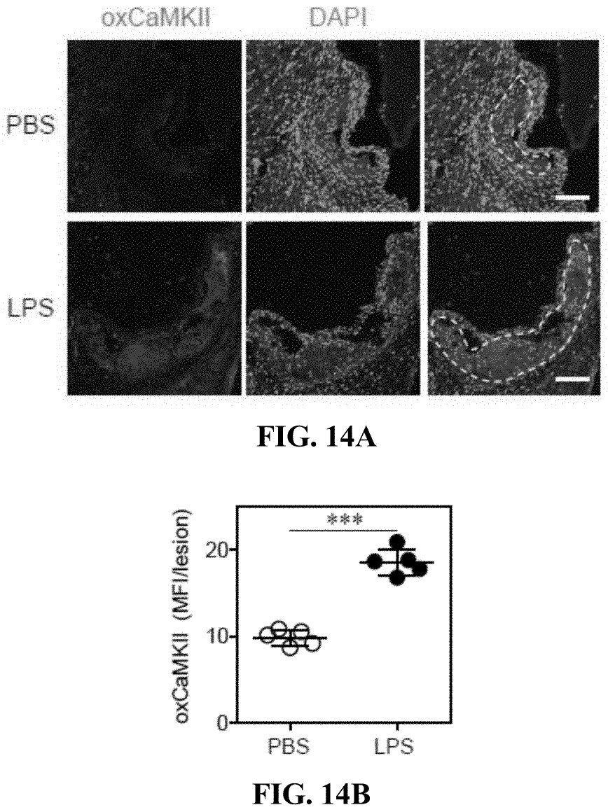

Given the finding of polarized inflammatory neutrophils both in vitro and in vivo by subclinical dose LPS, the potential underlying mechanism was examined. Since activation of 5-LOX mediated by oxCAMKII have been shown to be important for the expression of inflammatory mediators such as LTB4, the activation status of oxidized calmodulin-dependent protein kinase II (oxCAMKII) and 5-lipoxygenase (5-LOX) in neutrophils challenged with subclinical dose LPS was examined. As shown in FIG. 3D, it was observed that neutrophils cultured with subclinical dose LPS had increased levels of oxCAMKII and 5-LOX. Through in situ immune-histochemical staining, it was further confirmed that the levels of oxCAMKII were significantly elevated in either RD-fed or HFD-fed ApoE−/− mice chronically injected with low dose LPS as compared to corresponding control mice with PBS injection (FIGS. 13A-13B and 14A-14B).

OxCAMKII mediated signal transducer and activator of transcription 1 (STAT1) activation is responsible for the expression of Dectin-1 (30, 31). Therefore, the activation status of STAT1 was measured through detecting the levels of phosphorylated STAT1 (p-STAT1) by flow cytometry, and observed significantly elevated levels of p-STAT1 in neutrophils challenged with subclinical dose LPS (FIG. 3E). On the other hand, homeostatic transcription factors such as Kruppel-like factor-2 (KLF2) and activating transcription factor 4 (ATF4) are involved in transcribing homeostatic molecules such as FPN and LRRC32, as well as reducing ROS-mediated activation of oxCAMKII (32-35). It was observed that subclinical dose LPS significantly reduced the cellular levels of KLF2 and ATF4 in neutrophils (FIG. 3F). Collectively, the activation of oxCAMKII and the reduction of homeostatic KLF2/ATF4 may be responsible for polarizing neutrophils into the non-resolving inflammatory state conducive to atherosclerosis.

### In Vitro Polarized Non-Resolving Inflammatory Neutrophils is Sufficient to Confer Plaque Instability.

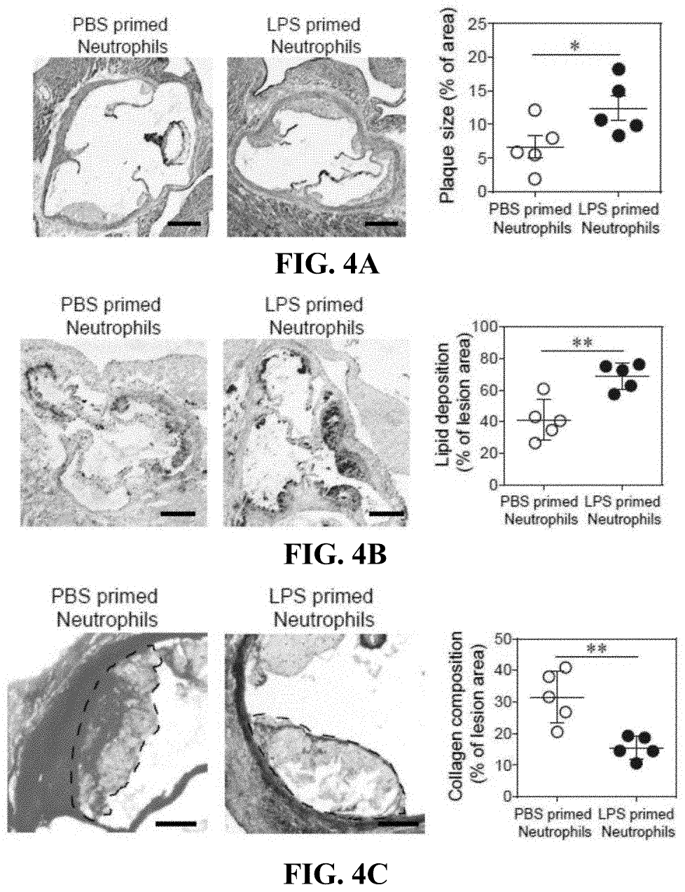

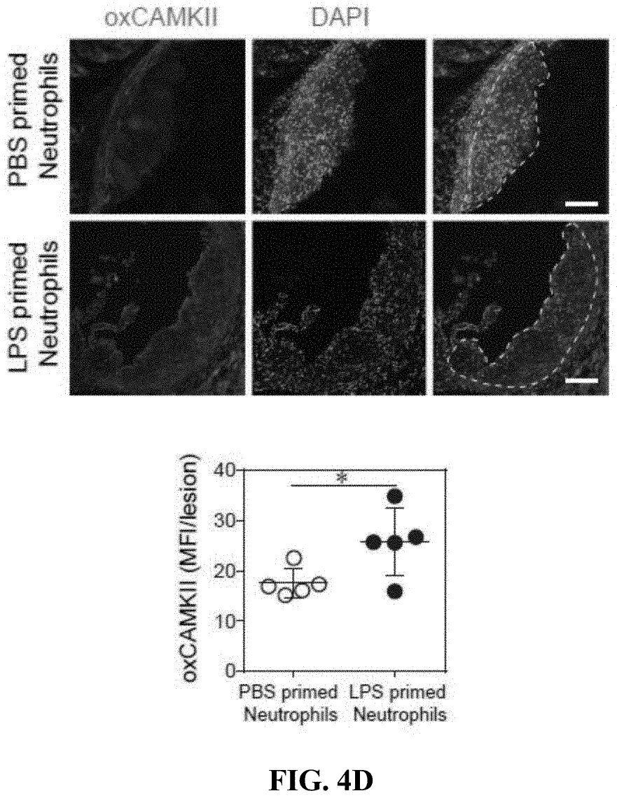

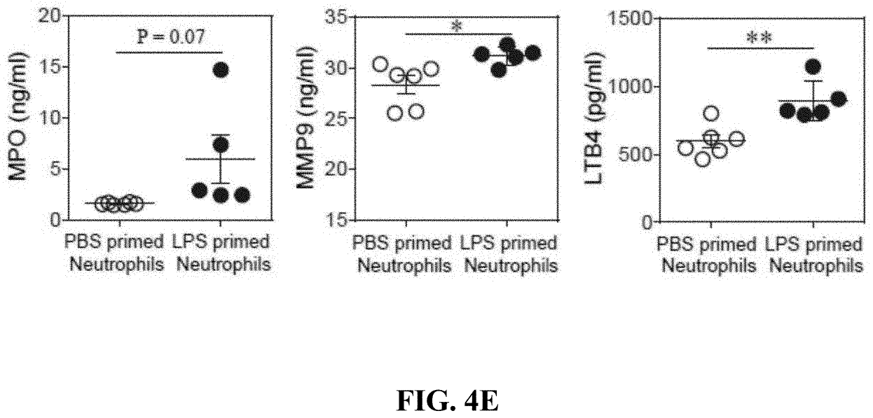

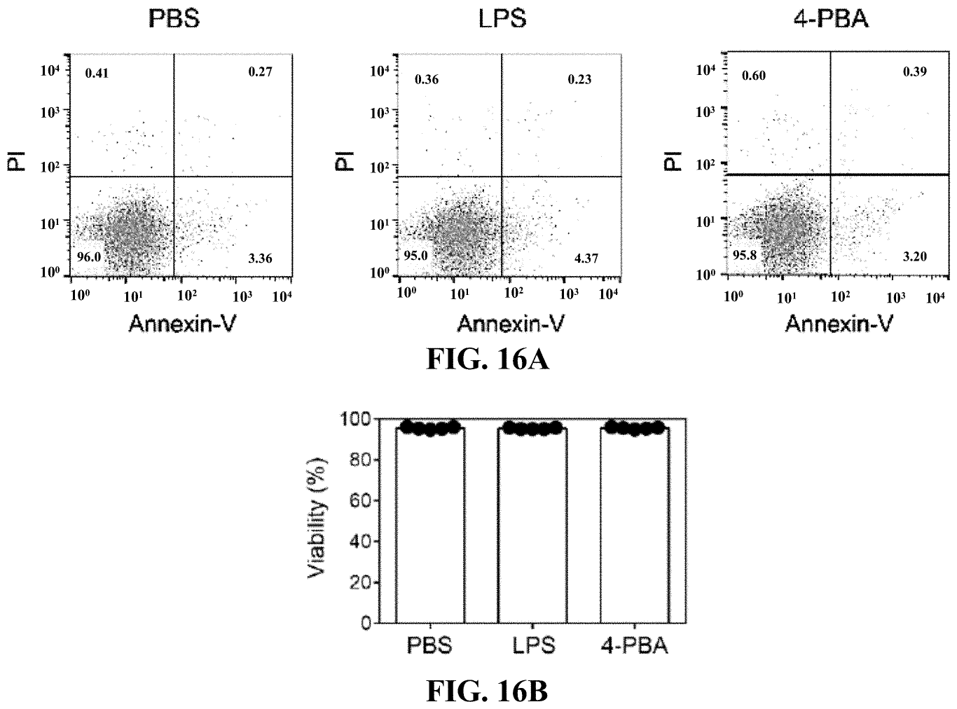

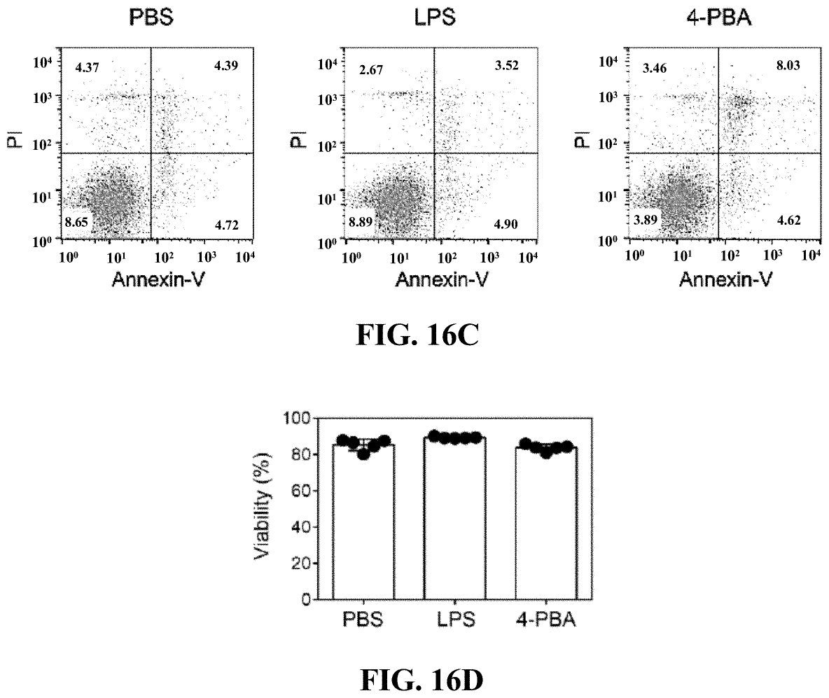

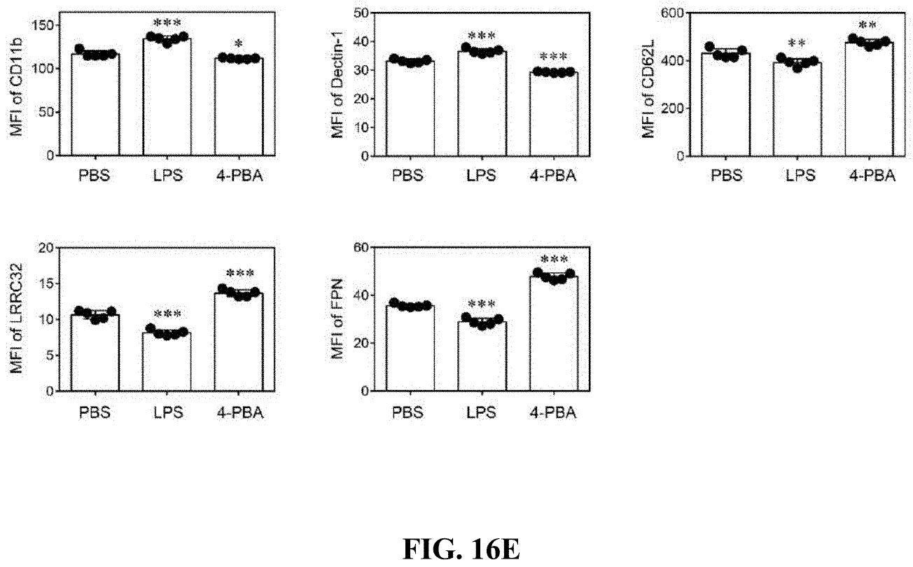

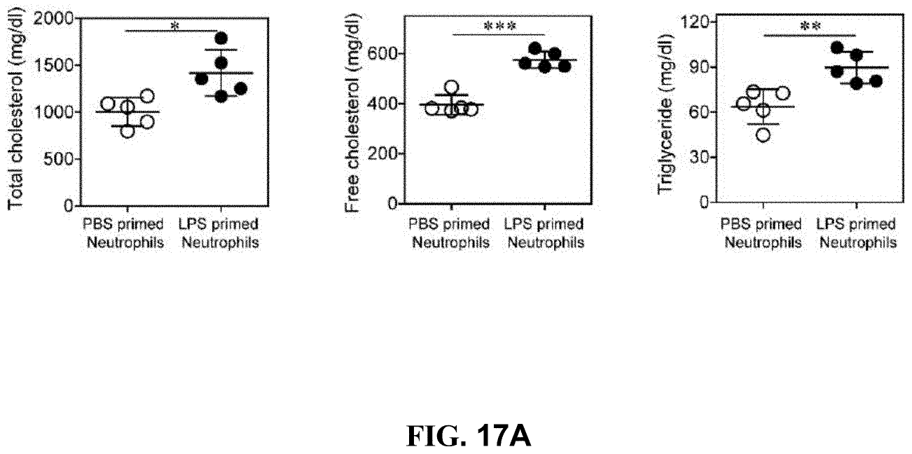

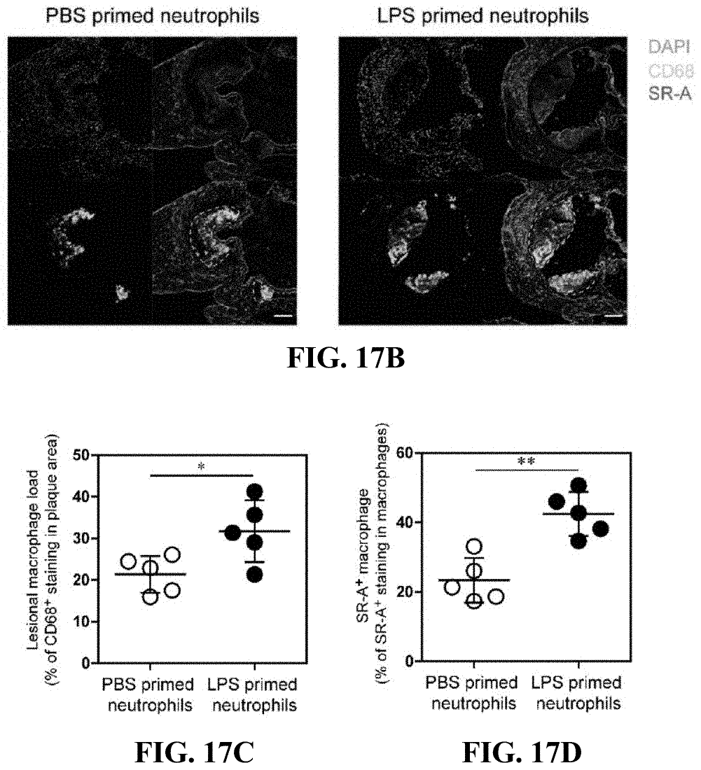

In order to test whether polarized neutrophils by subclinical dose LPS is directly responsible for exacerbated atherosclerosis and plaque instability, we performed experiments by transfusing in vitro polarized neutrophils into recipient animals. Bone-marrow neutrophils were polarized through in vitro culture with G-CSF plus either low dose LPS or PBS overnight. HFD-fed ApoE−/− mice were injected via intravenously with neutrophils cultured in vitro by either PBS or LPS on a weekly basis for one month. The viability of transferred neutrophils was determined by annexin V/PI (propidium iodide) staining, and the results demonstrated that >95% of cultured neutrophils remained viable after in vitro polarization for 24 hours (FIG. 16A-16B) and about 90% viable for 48 hours (FIGS. 16C-16D), consistent with other reports showing that murine BM neutrophils have a longer life span than circulating neutrophils and G-CSF delays neutrophil apoptosis (27, 28). Moreover, we tested the surface phenotype of the neutrophils before transfer. The neutrophils polarized by low-dose LPS for 24 hours exhibited elevated expressions of CD11b and Dectin-1 and reduced expressions of LRRC32, FPN, and CD62L (FIG. 16E). One week post the final injection, mice were sacrificed and examined. It was observed that mice injected with LPS-polarized neutrophils had significantly larger plaque sizes as well as higher lipid deposition within the plaques (FIGS. 4A and 4B). The collagen contents within the plaques from mice injected with LPS-polarized neutrophils were significantly lower as compared to mice injected with PBS-treated neutrophils (FIG. 4C). The status of key enzymes, such as oxCAMKII, responsible for generating inflammatory mediators within the plaques through immunohistochemical staining was tested, and it was observed a significantly elevated signal of oxCAMKII within the plaques of mice transfused with LPS-polarized neutrophils as compared to mice transfused with PBS-treated control neutrophils (FIG. 4D). Adoptive transfer of LPS-primed neutrophils also resulted in the elevation of total macrophage load and the frequency of SR-A+ macrophages in atherosclerotic plaques, suggesting that primed neutrophils may communicate with monocyte/macrophage population and induce macrophage activation, further amplifying the detrimental effect (FIGS. 17B-17D). Plasma samples were collected 1 week after the last neutrophil transfer, and the levels of total cholesterol, free cholesterol and triglycerides were determined (FIG. 17A). Consequently, it was observed significantly higher levels of plasma MPO, LTB4 and MMP9 in mice transfused with LPS polarized neutrophils as compared to mice transfused with PBS-treated control neutrophils (FIG. 4E). These data can at least demonstrate that polarized neutrophils by subclinical dose LPS is directly responsible for exacerbated atherosclerosis in vivo.

### Polarized Inflammatory Neutrophils Exhibit Disrupted Peroxisome Homeostasis and Elevated ROS.

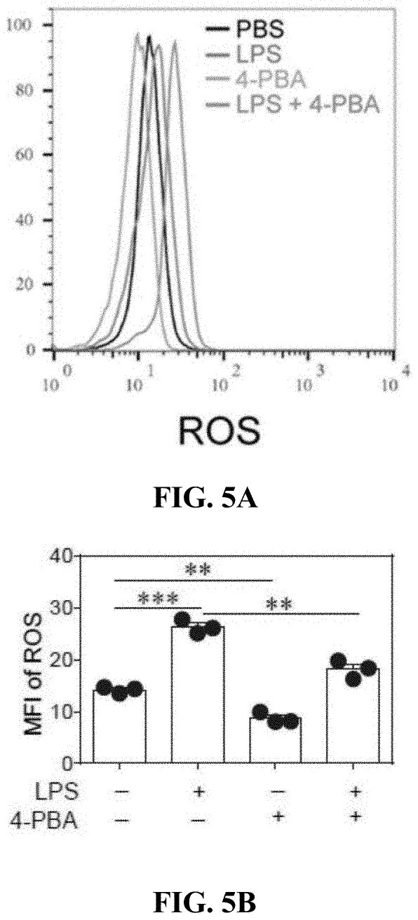

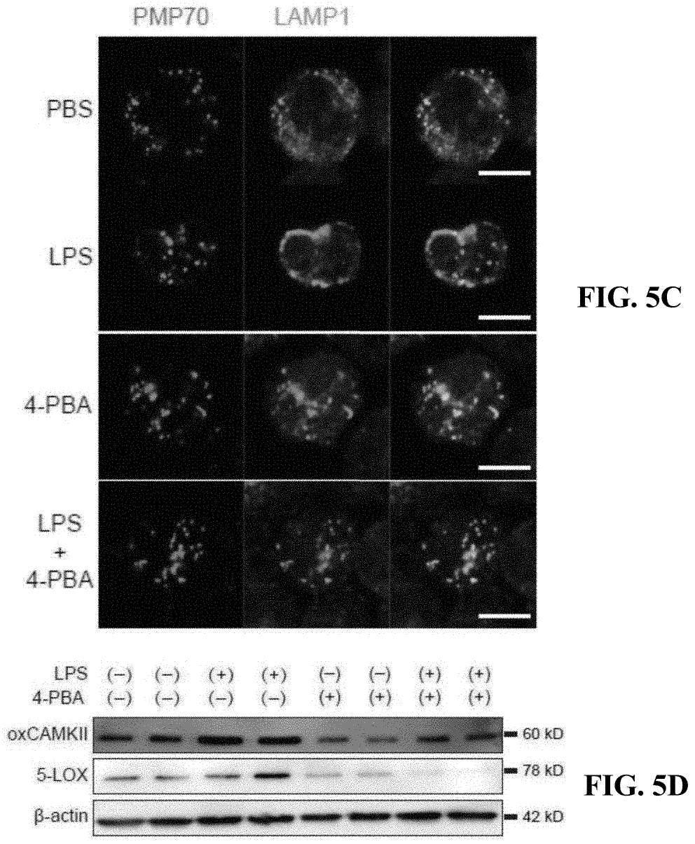

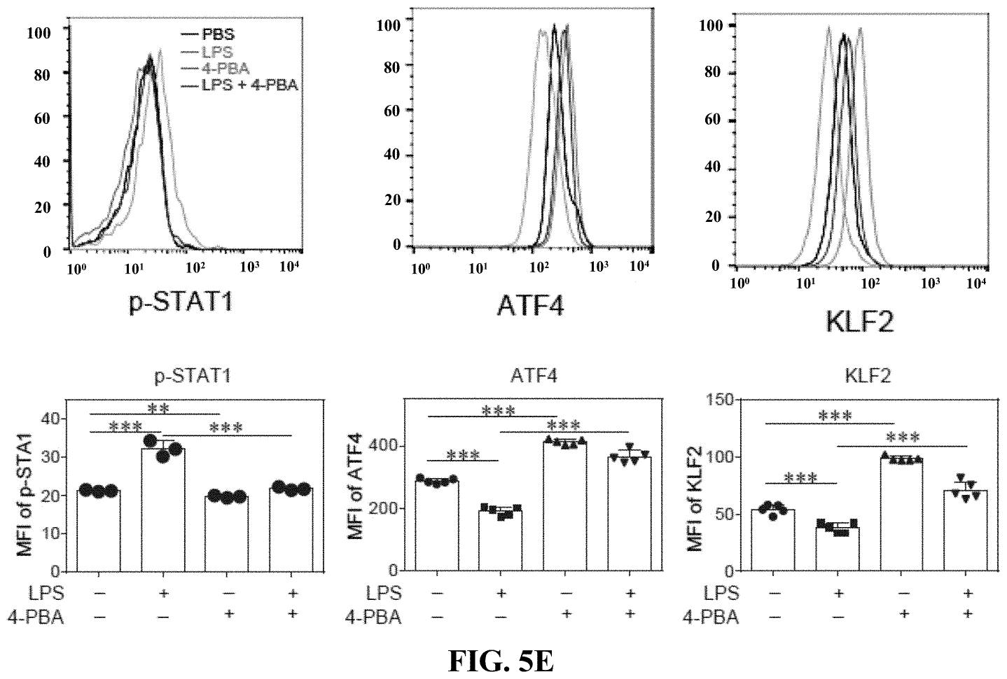

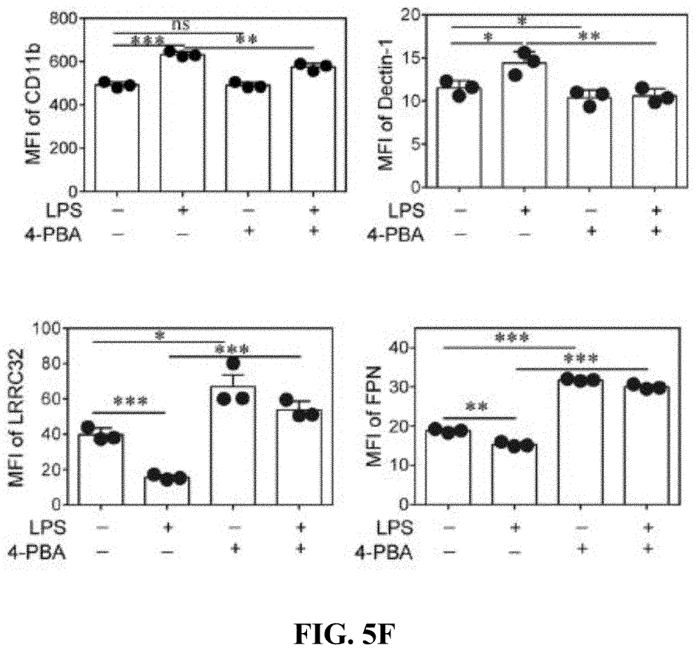

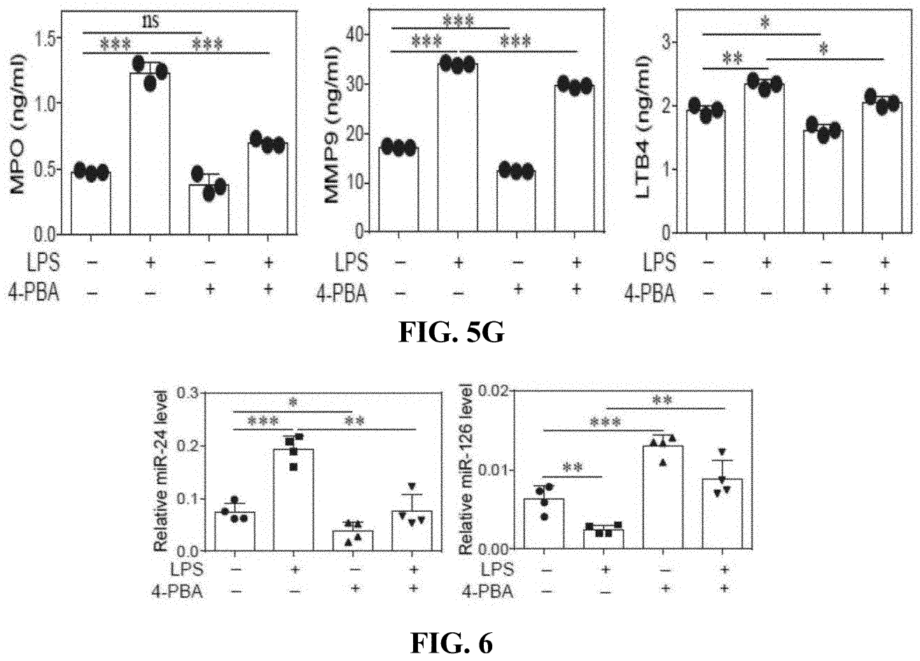

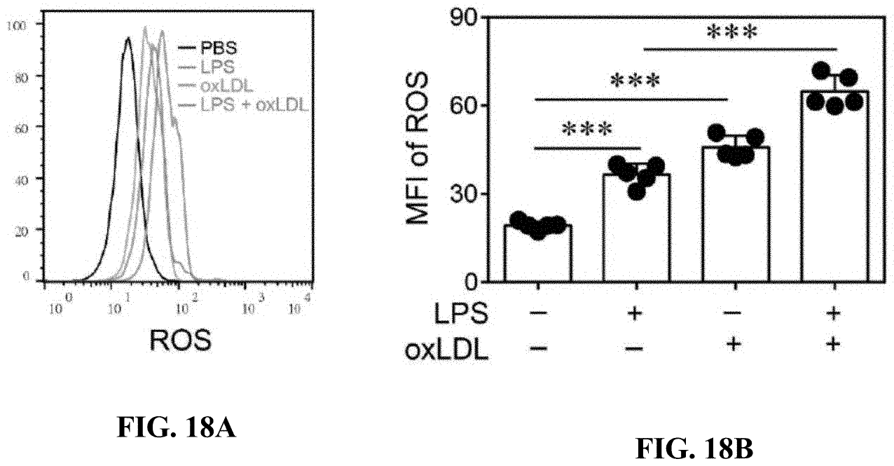

The data reveal a previously unknown aspect of neutrophil polarization that is conducive for exacerbated atherosclerosis. This finding complements emerging studies that suggest neutrophils may be differentially programmed into distinct functional state with significant pathophysiological implications (36). However, cellular and molecular mechanisms underlying neutrophil polarization remain less studied. Given the above observation that neutrophils programmed by subclinical dose LPS exhibit elevated oxCAMKII, a critical signaling molecule involved in the expression of inflammatory mediators (37), it was further examined whether reactive oxygen species (ROS) were involved. The levels of intracellular ROS in neutrophils cultured with PBS and LPS was measured through staining with a ROS-specific fluorescent probe followed by flow cytometry. It was observed that neutrophils programmed with subclinical dose LPS exhibited significantly higher levels of ROS as compared to control neutrophils cultured with PBS (FIGS. 5A and 18A-18B). Altered peroxisome homeostasis is critically important for ROS generation, and it was thus examined peroxisome communication with lysosome within neutrophils. As shown in FIG. 5C, neutrophils cultured with subclinical dose LPS exhibited a disruption of proper fusion between peroxisome and lysosome. In contrast, PBS cultured control neutrophils exhibited efficient fusion of peroxisome with lysosome.

To test whether the disruption of peroxisome homeostasis is responsible for LPS-mediated neutrophil polarization, we employed a selective compound 4-PBA that is known to induce effective peroxisome homeostasis (38). As shown in FIG. 5C, application of 4-PBA effectively restored the fusion of peroxisome and lysosome in cells treated with LPS. Furthermore, 4-PBA challenge effectively ameliorated the induction of ROS by LPS in neutrophils (FIGS. 5A and 5B). The activation of oxCAMKII as well as the induction of 5-LOX by LPS were also effectively ameliorated by the addition of 4-PBA (FIG. 5D). 4-PBA incubation also led to a reduction of pSTAT1 and restoration of KLF2/ATF4 in neutrophils challenged with LPS (FIG. 5E). These data can demonstrate at least that these altered peroxisome homeostasis in neutrophils programmed by subclinical dose LPS is responsible for elevated ROS and inflammatory polarization, and that restoration of peroxisome homeostasis may hold promise for maintaining proper neutrophil function and alleviating atherosclerosis progression.

### 4-PBAA Restores Neutrophil Homeostasis.

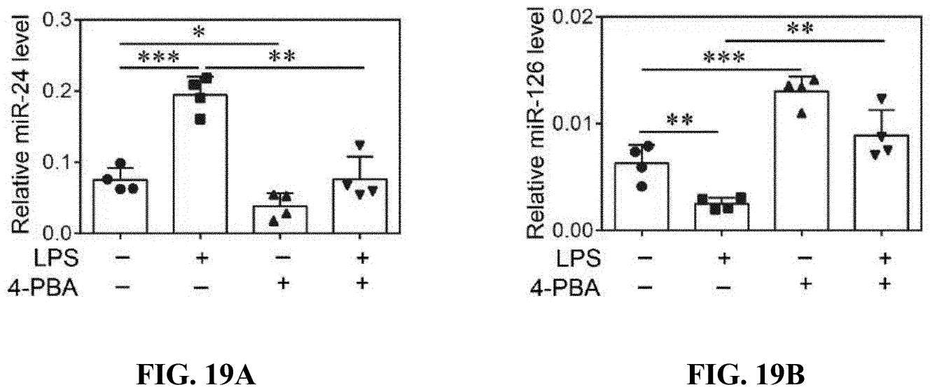

Given the finding that 4-PBA can effectively ameliorate LPS-induced induction of ROS and activation of oxCAMKKII, a functional test was performed to examine whether 4-PBA can restore functional homeostasis of neutrophils. To test this, the levels of cell surface markers, expressed inflammatory mediators, as well as key miRNAs involved in vascular inflammatory and homeostasis. As shown in FIG. 5F, 4-PBA effectively reduced the expression of inflammatory neutrophil surface markers such as CD11b, Dectin-1 induced by LPS. 4-PBA also restored the surface levels of CD62L in neutrophils (FIG. 5F). The addition of 4-PBA is sufficient to induce homeostatic surface markers such as FPN and LRRC32 (FIG. 5F). Through ELISA, it was observed that 4-PBA can potently reduce the induction of MPO, LTB4, and MMP9 in neutrophils by LPS (FIG. 5G). In terms of the miRNAs, 4-PBA treatment reduced the induction of pro-inflammatory miR-24 by LPS, and restored the induction of homeostatic miR-126 (FIGS. 6 and 19A-19B). These data can at least demonstrate that 4-PBA can effectively restore neutrophils homeostasis.

### Enhanced Neutrophil Homeostasis Alleviates Atherosclerosis.

Based on the observation that 4-PBA can potently enhance neutrophil homeostasis, it was tested whether enhanced homeostatic neutrophils by 4-PBA is sufficient to alleviate atherosclerosis. ApoE−/− mice fed with high fat diet were transfused weekly for 4 weeks with neutrophils cultured with either PBS or 4-PBA. The neutrophils polarized with 4-PBA for 24 hours demonstrated reduced CD11b and Dectin-1 expression and elevated LRRC32, FPN, and CD62L ex-pression (fig. S6E), exhibiting a similar phenotype as the neutrophils primed with 4-PBA for 48 hours. One week after the final transfusion, mice were harvested for analyses.

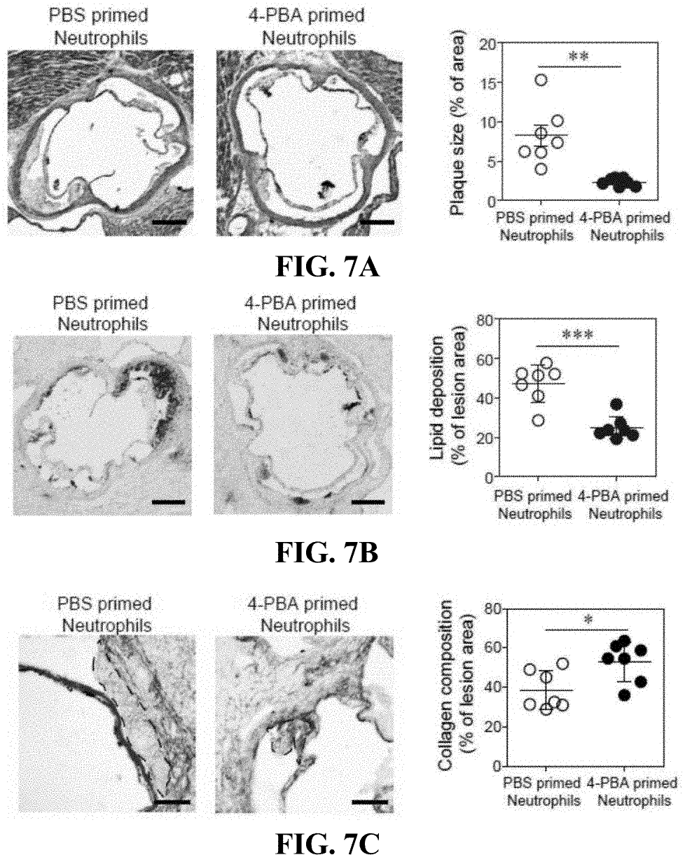

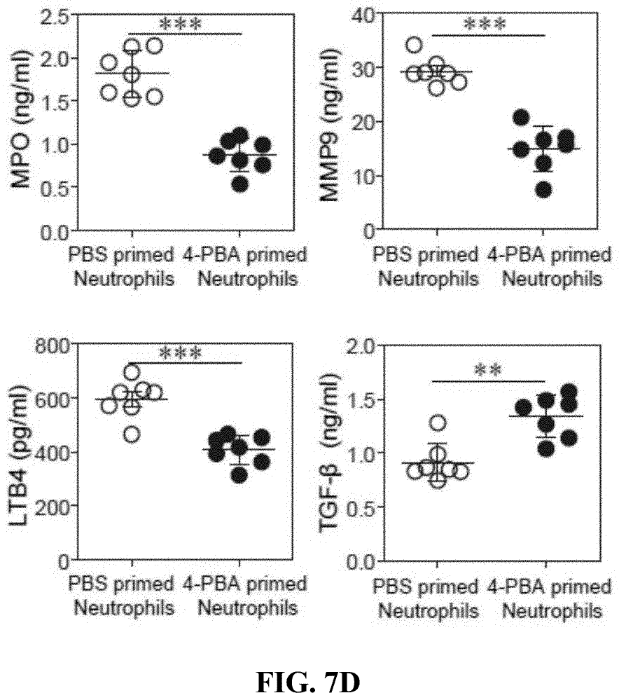

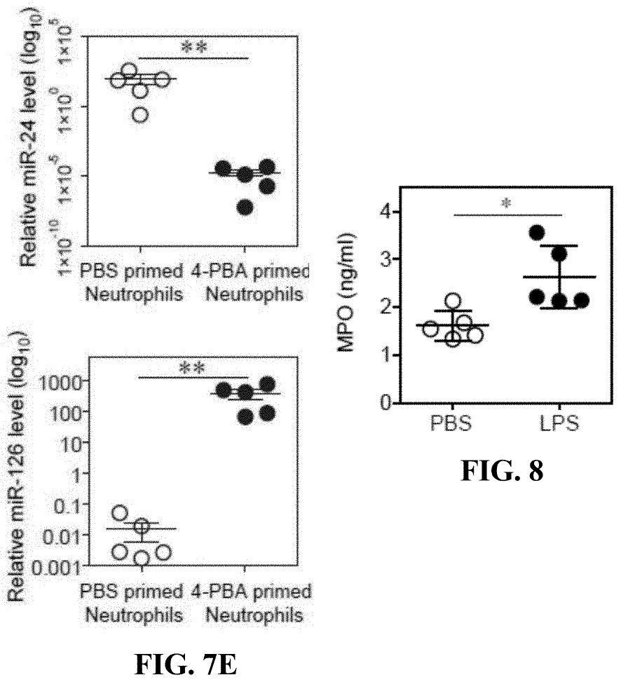

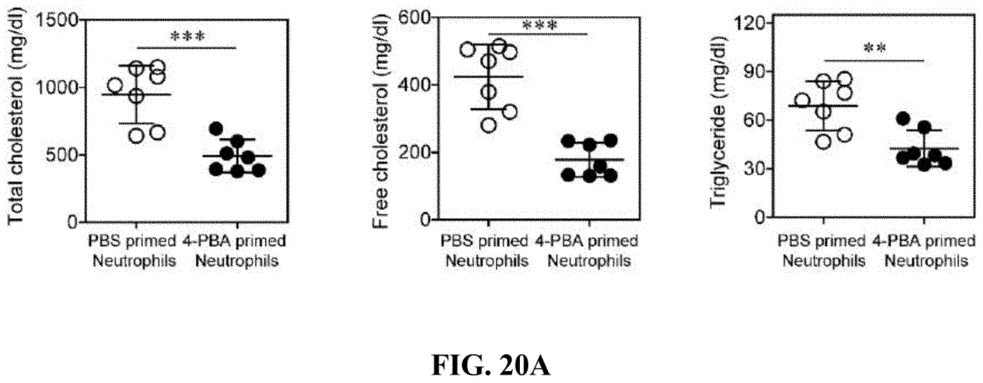

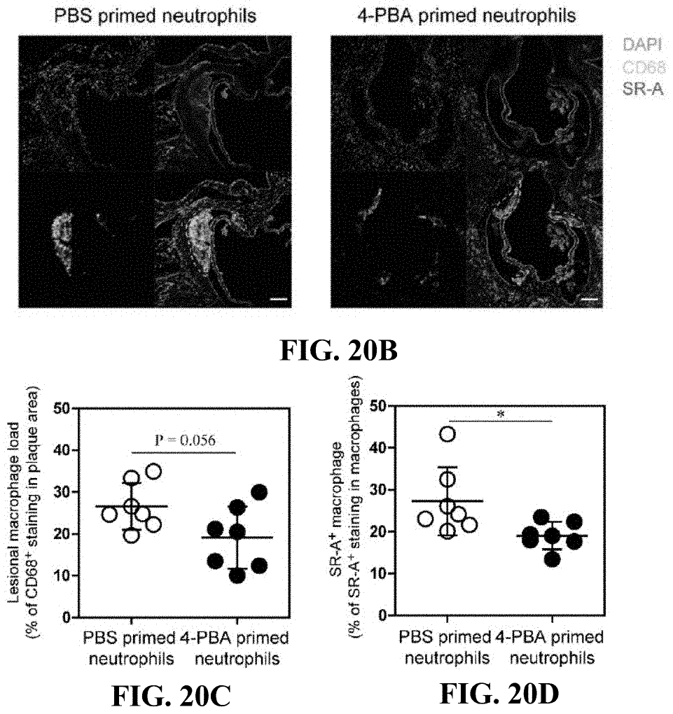

It was observed that mice transfused with 4-PBA programmed neutrophils had a 3-fold reduction in plaque sizes and a two-fold reduction of the plaque lipid content (FIGS. 7A and 7B). Collagen staining also revealed that mice transfused with 4-PBA programmed neutrophils had significantly higher levels of plaque collagen as compared to mice transfused with control neutrophils (FIG. 7C). Mice transferred with 4-PBA-polarized neutrophils exhibited a significant reduction of plasma cholesterol and triglycerides as compared to mice transferred with PBS-treated neutrophils (FIG. 20A). Furthermore, we observed a decline in activated SR-A+ macrophages within the plaques of mice transferred with 4-PBA-polarized neutrophils FIGS. 20A-20D).

In addition to significantly reduced plaque sizes, inflammatory markers were further measured in the experimental mice. It was observed that mice transfused with 4-PBA programmed neutrophils had significantly lower plasma levels of MPO, LTB4 and MMP9, as well as significantly elevated levels of TGFβ (FIG. 7D). As compared to mice transfused with control neutrophils, mice transfused with 4-PBA programmed neutrophils had significantly lower pro-inflammatory miR-24, and higher homeostatic miR-126 in circulation (FIG. 7E). These data can at least demonstrate that 4-PBA programmed neutrophils can be effectively used to restore tissue homeostasis in vivo, and hold promising potential to treat atherosclerosis.

Discussion.

This Example can at least demonstrate programming dynamics of neutrophils involved in the generation of unstable atherosclerotic plaques. We showed that pathologically-relevant super-low dose endotoxin can distinctly program neutrophils into a state with altered balance of non-resolving inflammation that is conducive to the development of unstable atherosclerotic plaques. The nature of non-resolving inflammatory neutrophils is reflected in elevated ROS due to disrupted peroxisome homeostasis, resulting in the skewed activation of oxCAMKII and downstream expression of LTB4, MMP9, as well as miR24, and reduced ATF4/KLF2-mediated expression of resolving mediators such as LRRC32 and miR126. We further demonstrated that the restoration of neutrophil peroxisome homeostasis through the application of 4-PBA can effectively resolve neutrophil inflammation, and that re-programmed neutrophils by 4-PBA can potently attenuate atherosclerosis progression.

This Example can at least demonstrate programming dynamics of neutrophils involved in the generation of unstable atherosclerotic plaques. This Example can at least demonstrate that pathologically-relevant super-low dose endotoxin can distinctly program neutrophils into a state with altered balance of non-resolving inflammation that is conducive to the development of unstable atherosclerotic plaques. The nature of non-resolving inflammatory neutrophils is reflected in elevated ROS due to disrupted peroxisome homeostasis, resulting in the skewed activation of oxCAMKII and downstream expression of LTB4, MMP9, as well as miR24, and reduced ATF4/KLF2-mediated expression of resolving mediators such as LRRC32 and miR126. It was further demonstrated that the restoration of neutrophil peroxisome homeostasis through the application of 4-PBA can effectively resolve neutrophil inflammation, and that re-programmed neutrophils by 4-PBA can potently attenuate atherosclerosis progression.

The data demonstrated here can fill this important void and can at least demonstrate, in addition to the elevated counts of neutrophils, the quality of polarized neutrophils may bear more significant relevance to atherosclerosis. It was observed that mice subjected to chronic injection of pathologically relevant subclinical-low dose LPS developed atherosclerotic plaques with elevated lipid deposition and reduced collagen content, characteristic of unstable plaques. It was observed that neutrophils underwent unique polarization in mice injected with low-dose LPS and adopted a skewed non-resolving inflammatory state represented by increased expression of pro-inflammatory mediators such as LTB4, MPO and MMP9 known to be major risk factors for unstable plaques (18-21, 40), as well as reduced expression of resolving mediators such as LRRC32 and miR126. LRRC32 is responsible for the generation of mature TGFβ and miR126 is critically involved in facilitating vascular integrity (26, 28, 29). Through adoptive transfer studies, it was demonstrated that the transfer of neutrophils polarized by super-low dose LPS was sufficient to significantly aggravate atherosclerosis and reduce plaque stability, as compared to the transfer of equal numbers of PBS-treated control neutrophils. This Example complements a recent report that the reduction of LRRC32 levels on human immune cells is associated with reduced human atherosclerotic plaque stability (41).

This data can at least demonstrate programming dynamics of neutrophils under chronic low-grade inflammatory conditions, and are consistent with a recent independent study regarding the effects of endotoxemia on plaque destabilization (21). Mawhin et al recently reported that mice repeatedly injected with 1.5 mg/kg LPS (˜30-60 μg/mouse) exhibited elevated numbers of circulating neutrophils potentially mediated by elevated LTB4, and severe atherosclerotic vulnerability with reduced collagen content and enlarged necrotic cores (21). However, that study did not define the fundamental nature of neutrophils affected by LPS and did not provide a causal connection between neutrophils and plaque instability. This Example not only complements the phenotypic observation of unstable plaques elicited by LPS injection, but also provides compelling mechanistic principles for neutrophil polarization directly responsible for the development of unstable plaques. We provided both in vitro and in vivo data that define the unique skewing of neutrophils by very-low dose LPS into a non-resolving inflammatory state, directly responsible for unstable atherosclerosis. This Example also better resembles the pathological conditions in humans and experimental animals with endotoxemia (42-46). Circulating endotoxin levels in humans and experimental animals are extremely low and within the picogram-nanogram/kg ranges, which are thousands-fold less than the micrograms to milligrams/kg range that most studies used (47-52).

The unique adaptations of innate leukocytes to varying dosages of bacteria endotoxin are highly complex and bears profound pathological relevance (53-58). It is believed that this Example provides the first characterization of unique neutrophil polarization by pathologically relevant concentration of endotoxin. It was observed that neutrophils are skewed to a polarized inflammatory state with reduced ability to homeostatic resolution. The identified features of polarized neutrophils such as elevated surface Dectin-1 and CD11b, reduced surface LRRC32 and FPN, as well as increased secretion of MPO, LTB4, miR-24 and reduced secretion of miR126 may serve as potential markers for characterizing neutrophils involved in various chronic inflammatory diseases such as atherosclerosis.

The qualitative natures, rather than simple elevation of neutrophil counts, have been increasingly implicated in the pathogenesis of other chronic diseases such as cancer (59, 60). Although neutrophils are known to be pleiotropic in nature, the exact characterization of neutrophil polarization and the underlying mechanisms are poorly defined. Filling this critical gap, this Example can at least demonstrate that the novel disruption of peroxisome homeostasis within neutrophils may be one of the critical culprits. We observed that polarized neutrophils by super-low dose endotoxin exhibit a disruption of proper fusion among peroxisomes and lysosomes, which contributes to the accumulation of ROS and the development of non-resoling inflammatory neutrophils. The accumulation of ROS is associated with elevated oxCAMKII responsible for the expression of LTB4, MPO and Dectin-1. On the other hand, polarized neutrophils have reduced ATF4/KLF2, key transcription factors beneficial for the resolution of inflammation through the expression of homeostatic molecules such as LRRC32 and miR126. ATF4/KLF2 are also known to closely cooperate with NRF2 in reducing ROS and restoring cellular homeostasis. Collectively, this Example can at least demonstrate and can define the disrupted molecular balance in neutrophils leading to the accumulation of ROS and the development of non-resolving inflammatory state conducive for atherosclerosis.

This Example can further demonstrate at least a novel and effective strategy in re-balancing the polarized neutrophils back to the homeostatic state through the application of 4-PBA. This Example can provide unique steps and features above and beyond previous biochemical characterizations of 4-PBA in other cells which demonstrated that 4-PBA can restore peroxisome homeostasis, among others (38, 61-65). It was observed that 4-PBA can restore peroxisome-lysosome fusion in neutrophils, and reduce LPS-mediated elevation of neutrophil ROS. 4-PBA can effectively reduce the induction of oxCAMKII, LTB4, MPO, Dectin-1, CD11b, miR-24 in neutrophils by super-low dose LPS, and also restore the expression of ATF4, FPN, LRRC32, and miR-126 in neutrophils suppressed by LPS. Functionally, it was demonstrated that rejuvenated homeostatic neutrophils in vitro by 4-PBA treatment can potently reduce the pathogenesis of atherosclerosis, as reflected in drastically reduced lesion sizes and elevated collagen contents.

Together, this Example not only provides compelling mechanistic data that address a unique aspect of neutrophil polarization relevant to the pathogenesis of unstable atherosclerotic plaques, but also demonstrates the novel feasibility of using re-programmed neutrophils to directly treat experimental atherosclerosis.

### Materials and Methods

Experimental animals and LPS injection. Male ApoE−/− mice (6 to 8-week old) on the C57 BL/6 background were purchased from the Jackson Laboratory and fed with regular diet or High Fat Diet. Either PBS or subclinical dose LPS (5 ng per kg body weight) was intraperitoneally injected every 3 days for 1 month. Then, the mice were sacrificed and tissues were harvested for further analyses. All animal procedures were in accordance with the US National Institutes of Health Guide for the Care and Use of Laboratory Animals and approved by the Intuitional Animal Care and Use Committee of Virginia Tech.

Analyses of neutrophil phenotype in vivo. Peripheral blood cells, bone marrow (BM) cells and splenocytes were harvested from the mice treated as described above. Cells were stained with anti-Ly6G (BioLegend, #127618, 1:200 dilution), anti-CD11b (BioLegend, #101206, 1:200 dilution), anti-CD62L (BioLegend, #104407, 1:200 dilution), anti-Dectin1 (BioLegend, #144305, 1:200 dilution), anti-LLRC32 (BioLegend, #142904, 1:200 dilution) and anti-FPN (Novus, #NBP1-21502, 1:200 dilution) antibodies. The surface phenotype of Ly6G+ neutrophils was analyzed by FACSCanto II (BD Biosciences). The data were processed by Flow Jo (Tree Star).

Adoptive transfer of in vitro neutrophils. Bone marrow (BM) cells were isolated from ApoE−/− mice and BM neutrophils (Ly6G+ population) were purified by FACS sorting with >99.5% purity. The cells were cultured in complete RPMI with G-CSF (100 ng/ml) in the presence of subclinical dose LPS (100 pg/ml) or 4-PBA (1 mM) for 24 h. Cells were washed three times with PBS and suspended in PBS for injection. HFD-fed ApoE−/− mice were transfused with 2×106 neutrophils in 200 μl PBS once weekly through intravenous injection for 4 weeks. One week after the last cell transfer, mice were sacrificed and tissues were harvested for subsequent analyses.

Analyses of atherosclerotic lesions. Histological analyses were performed on fresh-frozen and optimal cutting temperature (OCT) compound-embedded proximal aortic sections (8 mm). Slides were fixed in 4% neutral buffered formalin for 5 min, followed by H&E or Oil Red O staining. Collagen staining was performed using the Picrosirius Red Stain Kit (Poly-Sciences) according to the manufacturer's instructions. The samples were observed under a light microscope. The percentages of total lesion area, lipid deposition, and collagen composition were calculated. For immunofluorescence staining, proximal aortic sections were fixed with 4% neutral buffered formalin for 5 min, permeabilized with 0.1% saponins, and blocked with 10% normal goat serum (Jackson ImmunoResearch) for 1 hour. To detect lesional neutrophils, the samples were stained with Alexa Fluor 647-conjugated anti-Ly6G antibody (1:50 dilution; BioLegend, no. 127610). To detect lesional macrophages, the samples were costained with eFluor 660-conjugated anti-CD68 (1:100 dilution; Thermo Fisher Scientific, no. 50-0681-82) and rabbit anti-SR-A (1:100 dilution; Thermo Fisher Scientific, no. PA5-22956) antibodies, followed by staining with DyLight 488-conjugated goat anti-rabbit immunoglobulin G (IgG) (1:100 dilution; Thermo Fisher Scientific, no. 35552) in the dark at room tem-perature for 1 hour. To detect oxCAMKII levels, the samples were stained with rabbit anti-oxCaMKII antibody (1:100 dilution; Millipore, no. 07-1387) at 4° C. overnight and then with DyLight 488-conjugated goat anti-rabbit IgG (1:100 dilution; Thermo Fisher Scientific, no. 35552) in the dark at room temperature for 1 hour. 4′,6-Diamidino-2-phenylindole was used to stain nucleus. The samples were observed under a confocal microscope.

In vitro neutrophil priming and FACS analysis. Bone marrow (BM) neutrophils were isolated from C57 BL/6 mice and cultured in complete RPMI medium containing 10% fetal bovine serum, 2 mM L-glutamine, 1% penicillin/streptomycin in the presence of G-CSF (100 ng/ml). PBS, subclinical dose LPS (100 pg/ml), oxLDL (10 μg/ml) or 4-PBA (1 mM) was added to cell cultures. After 2 days, the cells were harvested and stained with anti-Ly6G (BioLegend, #127606, 127610 or 127618, 1:200 dilution), anti-CD11b (BioLegend, #101206, 1:200 dilution), anti-CD62L (BioLegend, #104407, 1:200 dilution), anti-Dectin1 (BioLegend, #144305, 1:200 dilution), anti-LLRC32 (BioLegend, #142904, 1:200 dilution) and anti-FPN (Novus, #NBP1-21502, 1:200 dilution) antibodies. In some experiments, the cells were fixed and permeabilized using transcription factor phospho buffer set (BD Biosciences), and then stained with primary anti-p-STAT1 (Cell Signaling, #8009S, 1:50 dilution), anti-ATF4 (Proteintech, #10835-1-AP, 1:50 dilution), and anti-KLF2 (Novus, #NBP2-61812, 1:100 dilution) antibodies followed by staining with Alexa Fluor 488 conjugated goat anti-rabbit IgG (Abcam, #ab150077, 1:2000 dilution). Surface phenotype and transcription factor phosphorylation of Ly6G+ neutrophils were analyzed by FACSCanto II (BD Biosciences). For intracellular ROS detection, 5 μM CellROX Green Reagent (ThermoFisher) was added to neutrophil cultures 30 min before harvesting, and cells were analyzed by FACSCanto II (BD Biosciences). The data were processed by Flow Jo (Tree Star).

ELISA and determination of plasma lipids. For in vivo analyses, plasma was collected from the mice at time of sacrificing. For in vitro analyses, purified BM neutrophils were cultured in complete RPMI medium with G-CSF (100 ng/ml) in the presence of PBS, subclinical dose LPS (100 pg/ml), oxLDL (10 μg/ml) or 4-PBA (1 mM), and supernatant was collected after 2 d. ELISA kit of MPO was purchased from ThermoFisher, and ELISA kits of MMP9, LTB4 and TGF-β1 were purchased from R&D Systems. Cholesterol quantitation kit was purchased from Sigma-Aldrich, and triglyceride quantification kit was purchased from BioVision.

Immunoblotting. Purified BM neutrophils were cultured in complete RPMI medium with G-CSF (100 ng/ml) in the presence of PBS, subclinical dose LPS (100 pg/ml), oxLDL (10 μg/ml) or 4-PBA (1 mM), and total cell lysate was extracted on day 2. Protein samples were separated with SDS-PAGE and transferred to PVDF membranes, which were probed with anti-oxCaMKII (Millipore, #07-1387, 1:500 dilution), anti-5 LOX (Cell Signaling, #3289S, 1:500 dilution), and anti-β-actin (Santa Cruz, #sc-47778, 1:1000 dilution) primary antibodies followed by horseradish peroxidase-conjugated anti-rabbit IgG (Cell Signaling, #7074S, 1:1000 dilution) or anti-mouse IgG (Cell Signaling, #7076S, 1:1000 dilution) secondary antibodies. Images were developed by a chemiluminescence ECL detection kit (ThermoFisher).

Real-Time RT-PCR for detecting of miRNAs. For in vivo analyses, plasma was collected from the mice at time of sacrificing. Circulating miRNAs were isolated with miRNeasy Serum/Plasma kit (Qiagen). For in vitro analyses, purified BM neutrophils were cultured in complete RPMI medium with G-CSF (100 ng/ml) in the presence of PBS, subclinical dose LPS (100 pg/ml), or 4-PBA (1 mM), and miRNAs were isolated with miRNeasy mini kit (Qiagen) after 2 d. TaqMan miRNA Assay kits for detection of U6, miR-16, miR-24, and miR-126 were purchased from ThermoFisher. Real-time RT-PCR was performed following the manual. U6 expression was used as the internal control for miRNA expressions in neutrophil cultures, and miR-16 expression was used as the internal control for plasma miRNA expressions.

Confocal Microscopy. Purified BM neutrophils were cultured in complete RPMI medium with G-CSF (100 ng/ml) in the presence of PBS, subclinical dose LPS (100 pg/ml), or 4-PBA (1 mM) for 2 d. To determine lysosome-peroxisome fusion, the cells were fixed with 4% PFA, deposited on slides through cytospin, and permeabilized with 0.2% Trinton X-100. The cells were blocked and stained with primary rabbit anti-mouse PMP70 antibody (1:1000) supplied in SelectFX® Alexa Fluor® 488 Peroxisome Labeling Kit (ThermoFisher, #S34201), followed by staining with Alexa Fluor 488-goat anti-rabbit secondary antibody (1:1000) supplied in the kit. After extensive washing with PBS, the cells were then stained with Cy3-anti-LAMP1 antibody (Abcam, #Ab67283, 1:1000) and observed under a confocal microscope.

Statistical Analysis. Statistical analysis was performed using Prism 6 software (GraphPad Software, La Jolla, Calif.), and data were expressed as means±SEM. The significance of the differences was assessed by Student's t-test (for two groups) or one-way ANOVA (for multiple groups). P<0.05 was considered statistically significant.

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

Further attributes, features, and embodiments of the present invention can be understood by reference to the following numbered aspects of the disclosed invention. Reference to disclosure in any of the preceding aspects is applicable to any preceding numbered aspect and to any combination of any number of preceding aspects, as recognized by appropriate antecedent disclosure in any combination of preceding aspects that can be made. The following numbered aspects are provided:

1. A chemically programmed neutrophil comprising:

increased surface expression of CD62L as compared to a control, decreased gene and/or protein expression of CD11b, MMP-9, LTB4, MPO, and/or Dectin-1 as compared to the control, increased gene and/or protein expression of FPN, TGFβ, and/or LRRC32 as compared to the control, decreased activity of oxCAMKII as compared to the control, or any combination thereof, wherein the control is a neutrophil or population thereof having non-resolving inflammation phenotype.

2. The chemically programmed neutrophil of aspect 1, further comprising an exogenous gene.

3. The chemically programmed neutrophil of aspect 2, wherein the exogenous gene is a selectable marker or a suicide gene.

4. The chemically programmed neutrophil of any one of aspects 1-3, wherein the chemically programmed neutrophil was made by the method comprising:

contacting a neutrophil with 4-phenylbutyrate.

5. The chemically programmed neutrophil of aspect 4, wherein the concentration of 4-phenylbutyrate ranges from about 0.1 mM to about 10 mM.

6. The chemically programmed neutrophil of aspect 5, wherein the concentration of 4-phenylbutyrate is about 1 mM.

7. The chemically programmed neutrophil of any one of aspects 4-6, wherein the step of contacting occurs ex vivo.

8. The chemically programmed neutrophil of any one of aspects 4-6, wherein the step of contacting occurs in vivo.

9. A pharmaceutical formulation comprising:

a chemically programmed neutrophil or population thereof as in any one of aspects 1-8; and

a pharmaceutically acceptable carrier.

10. A method comprising:

administering a chemically programmed neutrophil as in any one of aspects 1-8 or a population thereof or a pharmaceutical formulation as in claim 9 to a subject.

11. The method of aspect 10, wherein the subject has or is suspected of having non-resolving inflammation and/or related diseases or conditions, including but not limited to atherosclerosis, cardiovascular disease, stroke, myocardial infarction, neurological disease, and/or a symptom thereof.

12. A method of treating ad/or preventing non-resolving inflammation and/or related diseases or conditions, including but not limited to atherosclerosis, cardiovascular disease, stroke, myocardial infarction, neurological disease, and/or a symptom thereof in a subject in need thereof, the method comprising administering a chemically programmed neutrophil as in any one of aspects 1-8 or a population thereof or a pharmaceutical formulation as in claim 9 to the subject in need thereof.

13. A method of reducing arterial plaque in a subject in need thereof, the method comprising:

administering a chemically programmed neutrophil as in any one of aspects 1-8 or a population thereof or a pharmaceutical formulation as in claim 9 to the subject in need thereof.

14. A method of chemically programming a neutrophil, the method comprising:

contacting a neutrophil with 4-phenylbutyrate.

15. The method of aspect 14, wherein the concentration of 4-phenylbutyrate ranges from about 0.1 mM to about 10 mM.

16. The method of aspect 15, wherein the concentration of 4-phenylbutyrate is about 1 mM.

17. The method of any one of aspects 14-16, wherein the step of contacting occurs ex vivo.

18. The method of any one of aspects 14-17, further comprising expressing an exogenous gene in the neutrophil.

19. The method of any one of aspects 14-18, further comprising the step of harvesting the neutrophils from a subject, wherein the step of harvesting occurs before the step of contacting.

20. The method of any one of aspects 14-19, further comprising the step of administering the chemically programmed neutrophil to the subject.

21. The method of any one of aspects 14-16, wherein the step of contacting occurs in a subject.

22. The method of aspect 21, further comprising the step of administering an amount of 4-PBA or a pharmaceutical formulation thereof to the subject, wherein the step of administering occurs before the step of contacting.

23. A pharmaceutical formulation for treating and/or preventing non-resolving inflammation and/or related diseases or conditions, including but not limited to atherosclerosis, cardiovascular disease, stroke, myocardial infarction, neurological disease, and/or a symptom thereof in a subject in need thereof comprising:

a therapeutically effective amount of 4-phenylbutyrate or pharmaceutically acceptable salt thereof; and

a pharmaceutically acceptable carrier.

24. A method of treating and/or preventing non-resolving inflammation and/or related diseases or conditions, including but not limited to atherosclerosis, cardiovascular disease, stroke, myocardial infarction, neurological disease, and/or a symptom thereof in a subject in need thereof, the method comprising: administering a pharmaceutical formulation as in aspect 23 to the subject in need thereof

What is claimed is:

1. A chemically programmed neutrophil comprising:
increased surface expression of CD62L as compared to a control, decreased gene and/or protein expression of CD11b, MMP-9, LTB4, MPO, and/or Dectin-1 as compared to the control, increased gene and/or protein expression of FPN, TGFβ, and/or LRRC32 as compared to the control, decreased activity of oxCAMKII as compared to the control, or any combination thereof, wherein the control is a neutrophil or population thereof having non-resolving inflammation phenotype.

2. The chemically programmed neutrophil of claim 1, further comprising an exogenous gene.

3. The chemically programmed neutrophil of claim 2, wherein the exogenous gene is a selectable marker or a suicide gene.

4. The chemically programmed neutrophil of claim 1, wherein the chemically programmed neutrophil was made by the method comprising:
contacting a neutrophil with 4-phenylbutyrate.

5. The chemically programmed neutrophil of claim 4, wherein the concentration of 4-phenylbutyrate ranges from about 0.1 mM to about 10 mM.

6. The chemically programmed neutrophil of claim 5, wherein the concentration of 4-phenylbutyrate is about 1 mM.

7. The chemically programmed neutrophil of claim 4, wherein the step of contacting occurs ex vivo.

8. The chemically programmed neutrophil of claim 4, wherein the step of contacting occurs in vivo.

9. A pharmaceutical formulation comprising:
a chemically programmed neutrophil or population thereof as in any one of claim 1; and
a pharmaceutically acceptable carrier.

10. A method comprising:
administering a chemically programmed neutrophil as in claim 1 or a population thereof or a pharmaceutical formulation thereof to a subject.

11. The method of claim 10, wherein the subject has or is suspected of having non-resolving inflammation and/or related diseases or conditions, including but not limited to atherosclerosis, cardiovascular disease, stroke, myocardial infarction, neurological disease, and/or a symptom thereof.

12. A method of treating and/or preventing non-resolving inflammation and/or related diseases or conditions, including but not limited to atherosclerosis, cardiovascular disease, stroke, myocardial infarction, neurological disease, and/or a symptom thereof in a subject in need thereof, the method comprising
administering a chemically programmed neutrophil as in claim 1 or a population thereof or a pharmaceutical formulation thereof to the subject in need thereof.

13. A method of reducing arterial plaque in a subject in need thereof, the method comprising:
administering a chemically programmed neutrophil as in claim 1 or a population thereof or a pharmaceutical formulation thereof to the subject in need thereof.

14. A method of chemically programming a neutrophil, the method comprising:
contacting a neutrophil with 4-phenylbutyrate.

15. The method of claim 14, wherein the concentration of 4-phenylbutyrate ranges from about 0.1 mM to about 10 mM.

16. The method of claim 15, wherein the concentration of 4-phenylbutyrate is about 1 mM.

17. The method of claim 14, wherein the step of contacting occurs ex vivo.

18. The method of claim 14, further comprising expressing an exogenous gene in the neutrophil.

19. The method of claim 14, further comprising the step of harvesting the neutrophils from a subject, wherein the step of harvesting occurs before the step of contacting.

20. The method of claim 14, further comprising the step of administering the chemically programmed neutrophil to the subject.

21. The method of claim 14, wherein the step of contacting occurs in a subject.

22. The method of claim 21, further comprising the step of administering an amount of 4-PBA or a pharmaceutical formulation thereof to the subject, wherein the step of administering occurs before the step of contacting.

23. A pharmaceutical formulation for treating and/or preventing non-resolving inflammation and/or related diseases or conditions, including but not limited to atherosclerosis, cardiovascular disease, stroke, myocardial infarction, neurological disease, and/or a symptom thereof in a subject in need thereof comprising:
a therapeutically effective amount of 4-phenylbutyrate or pharmaceutically acceptable salt thereof; and
a pharmaceutically acceptable carrier.

24. A method of treating and/or preventing non-resolving inflammation and/or related diseases or conditions, including but not limited to atherosclerosis, cardiovascular disease, stroke, myocardial infarction, neurological disease, and/or a symptom thereof in a subject in need thereof, the method comprising:
administering a pharmaceutical formulation as in claim 23 to the subject in need thereof.
